(19) United States
(12) Patent Application Publication
(10) Pub. No.: US 2022/0202557 A1
(43) Pub. Date: Jun. 30, 2022

(54) METHOD AND DEVICE FOR ACUTE TREATMENT OF FLUID OVERLOAD IN PATIENTS WITH HEART FAILURE

(71) Applicant: W. L. Gore & Associates, Inc., Newark, DE (US)

(72) Inventors: Kevin K. Coffman, Phoenix, AZ (US); Jason D. Hemmer, Newark, DE (US); Timothy E. Johnston, Flagstaff, AZ (US); Eric A. Mokelke, Flagstaff, AZ (US); Mark E. Trokanski, Phoenix, AZ (US)

(21) Appl. No.: 17/604,677

(22) PCT Filed: Apr. 16, 2020
(86) PCT No.: PCT/US2020/028533

§ 371 Date: Oct. 18, 2021

Related U.S. Application Data
(60) Provisional application No. 62/877,624, filed on Jul. 23, 2019.
(60) Provisional application No. 62/835,190, filed on Apr. 17, 2019.

(51) Int. Cl. A61F 2/07; A61B 17/12; A61B 5/026; A61B 5/00; A61F 2/82

(52) U.S. Cl. CPC A61F 2/07; A61B 17/12036; A61B 17/12109; A61B 17/12136; A61B 2017/00022; A61B 5/026; A61B 5/4836; A61B 5/6876; A61F 2/82; A61B 17/12168

(57) ABSTRACT

Various aspects of the present disclosure are directed toward apparatuses, systems and methods that include arranging an acute alteration of blood flow in, for example, heart failure patients.

### CROSS-REFERENCE TO RELATED APPLICATION

This application is a national phase application of PCT Application No. PCT/US2020/028533, internationally filed on Apr. 16, 2020, which claims the benefit of Provisional Application No. 62/877,624, filed Jul. 23, 2019, and also claims the benefit of Provisional Application No. 62/835,190, filed Apr. 17, 2019, both of which are incorporated herein by reference in their entireties for all purposes.

### TECHNICAL FIELD

The disclosure relates to systems, medical devices, and methods for treating heart failure and/or other cardiovascular diseases. More specifically, the disclosure relates to acute treatment by removing buildup of excess fluid that increases pressure on a patient's heart.

### BACKGROUND

Patients experiencing heart failure may have a buildup of excess fluid in the body. The excess fluid buildup may increase fluid accumulation in the interstitial space and add stress on an already failing heart. Excess fluid (or hypervolemia) is the leading cause of hospitalization for heart failure patients (approximately 1,000,000 per year in the United States).

Treatment of the excess fluid buildup may be treated pharmaceutically by diuretics (or other pharmaceutical agents). However, a patient may experience drug resistance, inaccurate dosing, or other issues such as failure to comply with medicine directives or diet. Non-pharmaceutical options, such as implantable or indwelling device solutions that provide an alternative to or augment pharmaceutical efficacy by influencing renal function, may be beneficial to avoid these and other issues in treatment of buildup of excess fluid in the body. Similarly, chronic high blood pressure (hypertension) can also be managed pharmaceutically by anti-hypertensive medications (or other pharmaceutical agents). In addition, other disease states may result in hypotension, reduced cardiac output, and poor renal function. Insofar as the kidneys play a central role in regulating systemic blood pressure and regulating sympathetic activity or tone, non-pharmaceutical options, such as implantable or indwelling device solutions that provide an alternative to or augment pharmaceutical efficacy by influencing renal function, may provide an alternative means of managing hypertension and other disease states.

### SUMMARY

According to one example (“Example 1”), an indwelling medical device for acute alteration of blood flow in a vessel of a patient includes a catheter; a vessel opposing member portion arranged at or extending from a distal end of the catheter and configured to oppose against a vessel wall of the vessel; and a flow altering element, arranged within the vessel opposing member portion and including a lumen for the blood flow through the vessel therethrough, configured to alter the blood flow through the lumen to restrict blood flow in the vessel and induce a physiologically mediated therapeutic response in the patient.

According to another example (“Example 2”), further to the device of Example 1, the vessel is the aorta and the vessel opposing member portion is configured to oppose against the vessel wall in the aorta and create a narrowed flow lumen in a conduit located in the aorta distal of one or both renal arteries of between about 40% and about 80% to alter blood flow into the at least one branch vessel of the aorta to alter blood flow into one or both kidneys.

According to another example (“Example 3”), further to the device of Example 1, the vessel is the vena cava and the vessel opposing member portion is configured to oppose against the vessel wall in the vena cava create a narrowed flow lumen in the conduit located in the vena cava distal of one or both renal veins of between about 40% and about 90% to and alter blood flow through one or both of the renal veins to alter blood flow into one or both kidneys.

According to another example (“Example 4”), further to the device of Example 3, the vessel opposing member portion is arranged upstream from one or both of the renal veins and the flow altering element is configured to drop blood pressure out of one or both of the renal veins to promote blood flow through the kidneys.

According to another example (“Example 5”), further to the device of any one of Examples 1-4, the flow altering element is at least one of a balloon, stent, stent-graft, sheath, and a restricting band, and vessel opposing member portion is comprised of at least one or more elements such as an outer balloon, a stent, a polymer material or other structures.

According to another example (“Example 6”), further to the device of Example 5, the flow altering element is a balloon, and the vessel opposing member portion is at least one of an outer balloon and further including a plurality of inflation ports configured to inflate and deflate the outer balloon and the flow altering element.

According to another example (“Example 7”), further to the device of Example 6, the outer balloon and the flow altering element are of substantially similar lengths.

According to another example (“Example 8”), further to the device of Example 5, a length of the flow altering element is less than a length of the outer balloon.

According to another example (“Example 9”), further to the device of any one of Examples 6-8, one of the plurality of ports is configured to provide flow to restrict an internal diameter of at least a portion of the outer balloon to alter the lumen.

According to another example (“Example 10”), further to the device of any one of Examples 6-8, the device also includes a plurality of struts coupled to the catheter and arranged within the inner or outer balloon, the plurality of struts being configured to support the balloon.

According to another example (“Example 11”), further to the device of any one of Examples 1-10, the lumen of the flow altering element includes at least one of a cylindrical shape, hourglass shape, a shape with circular restriction portions, a shape with stepped restriction portions, a ramped shape, and a peaked shape.

According to another example (“Example 12”), further to the device of any one of Examples 1-11, the flow altering element is at least one of a balloon, stent, stent-graft, restricting band, and sheath.

According to another example (“Example 13”), a method of increasing blood flow into one or both kidneys of the patient to increase urine production includes the device of any one of Examples 1-12.

According to one example (“Example 14”), an indwelling medical device for acute alteration of blood flow in an aorta or vena cava of a patient includes a catheter; a conduit, arranged at or extending from a distal end of the catheter, including a lumen for the blood flow through the aorta or vena cava; and at least one constraining fiber, arranged about a circumference of the conduit, configured to alter the blood flow through the lumen to restrict blood flow in the aorta or vena cava and alter blood flow into one or both kidneys of the patient in response to tension.

According to another example (“Example 15”), further to the device of Example 14, the conduit is configured to oppose against the vessel wall in the aorta and create a narrowed flow lumen in the conduit of the aorta distal of one or both renal arteries of between about 40% and about 80% to promote blood flow to the kidneys.

According to another example (“Example 16”), further to the device of Example 14, the conduit is configured to oppose against the vessel wall in the vena cava creating a narrowed flow lumen located in the vena cava distal of one or both renal veins, between about 40% and about 90% to promote blood flow to the kidneys.

According to another example (“Example 17”), further to the device of Example 16, the conduit is arranged upstream from one or both of the renal veins and the at least one constraining fiber is configured to drop blood pressure out of one or both of the renal veins to promote blood flow through the kidneys.

According to another example (“Example 18”), further to the device of any one of Examples 14-17, the at least one constraining fiber includes a plurality of constraining fibers arranged at different portions along a length of the conduit.

According to another example (“Example 19”), a method of increasing blood flow into one or both kidneys of the patient to increase urine production includes the device of any one of Examples 14-18.

According to one example (“Example 20”), an indwelling medical device for acute alteration of blood flow in an aorta or vena cava of a patient includes a catheter; a conduit, arranged at or extending from a distal end of the catheter, including a lumen for the blood flow through the aorta or vena cava; and a leaflet construct, arranged within the lumen of the conduit, configured to alter the blood flow through the lumen to restrict blood flow in the aorta or vena cava and alter blood flow into or out of one or both kidneys of the patient.

According to another example (“Example 21”), further to the device of Example 20, the conduit is configured to oppose against the vessel wall in the aorta and create a narrowed flow lumen of the aorta distal of one or both renal arteries of between about 40% and about 80% to promote blood flow to the kidneys.

According to another example (“Example 22”), further to the device of Example 20, the conduit is configured to oppose against the vessel wall in the vena cava and create a narrowed flow lumen of the vena cava distal of one or both renal veins of between about 40% and about 90% to promote blood flow into the kidneys.

According to another example (“Example 23”), further to the device of Example 22, the conduit is arranged upstream from one or both of the renal veins and the leaflet construct is configured to drop blood pressure out of one or both of the renal veins to promote blood flow through the kidneys.

According to another example (“Example 24”), further to the device of any one of Examples 20-23, further including one or more actuation members that are configured to open and close the leaflet construct.

According to another example (“Example 25”), further to the device of Example 24, the one or more actuation members includes a single actuation member configured to open and close each of the leaflets in the leaflet construct.

According to another example (“Example 26”), further to the device of Example 24, the one or more actuation members includes a number of actuation members equal to a number of leaflets in the leaflet construct.

According to another example (“Example 27”), further to the device of any one of Examples 24-26, the leaflet construct is coupled to hinge members arranged within the lumen to facilitate open and closed of the leaflet construct.

According to another example (“Example 28”), a method of increasing blood flow into or out of one or both kidneys of the patient to increase urine production includes the device of any one of Examples 20-28.

According to one example (“Example 29”), an indwelling medical device for acute alteration of blood flow in an aorta or vena cava of a patient includes an upper portion configured to oppose against a vessel wall of the aorta or vena cava; a lower portion configured to oppose against a vessel wall of the aorta or vena cava; a conduit, arranged between the upper portion and the lower portion, including a lumen for the blood flow through the aorta or vena cava; and a catheter configured to rotate at least one of the upper portion and the lower portion to alter the blood flow through the lumen to restrict blood flow in the aorta or vena cava and alter blood flow into one or both kidneys.

According to another example (“Example 30”), further to the device of Example 29, the upper portion and the lower portion are configured to oppose against the vessel wall in the aorta and create a narrowed flow lumen in the conduit located in the aorta, distal of one or both renal arteries of between about 40% and about 80% to increase blood flow to the kidneys.

According to another example (“Example 31”), further to the device of Example 29, the upper portion and the lower portion are configured to oppose against the vessel wall in the vena cava to create a narrowed flow lumen in the conduit located in the vena cava, distal of one or both renal veins of between about 40% and about 90% to and alter blood flow through one or both of the renal veins.

According to another example (“Example 32”), further to the device of Example 31, the upper portion and the lower portion are arranged upstream from one or both of the renal veins and configured to drop blood pressure out of one or both of the renal veins to promote blood flow through the kidneys.

According to another example (“Example 33”), further to the device of any one of Examples 29-32, further comprising a first plurality of struts coupled to the catheter and the upper portion configured to rotate the upper portion in response to rotation of the catheter, and a second plurality of struts coupled to the catheter and the lower portion configured to rotate the lower portion in response to rotation of the catheter.

According to another example (“Example 34”), further to the device of any one of Examples 29-33, the catheter is configured to independently rotate the upper portion and the lower portion.

According to another example (“Example 35”), further to the device of any one of Examples 29-34, the conduit is substantially cylindrical and includes a diameter less than a diameter of at least one of the upper portion and the lower portion.

According to another example (“Example 36”), a method of increasing blood flow into one or booth kidneys of the patient to increase urine production includes the device of any one of Examples 30-36.

According to another example (“Example 37”), a method of acute treatment of heart failure of a patient includes arranging an indwelling medical device coupled to a catheter within an aorta or vena cava of the patient; restricting blood flow through the indwelling medical device to induce a physiologically mediated therapeutic response by reducing buildup of excess fluid within the patient; and removing the indwelling medical device from the aorta or vena cava of the patient.

According to another example (“Example 38”), further to the method of Example 37, the indwelling medical device includes a vessel opposing member portion arranged at or extending from a distal end of the catheter and configured to oppose against a vessel wall of the aorta or vena cava and a flow altering element, arranged within the vessel opposing member portion and including a lumen for the blood flow through the aorta or vena cava therethrough, configured to alter the blood flow through the lumen to restrict blood flow in the aorta or vena cava and alter blood flow into one or both kidneys of the patient.

According to another example (“Example 39”), further to the method of Example 37, the indwelling medical device includes a conduit, arranged at or extending from a distal end of the catheter, including a lumen for the blood flow through the aorta or vena cava; and at least one constraining fiber, arranged about a circumference of the conduit, configured to alter the blood flow through the lumen to restrict blood flow in the aorta or vena cava and alter blood flow into one or both kidneys of the patient in response to tension.

According to another example (“Example 40”), further to the method of Example 37, the indwelling medical device includes a conduit, arranged at or extending from a distal end of the catheter, including a lumen for the blood flow through the aorta or vena cava; and a leaflet construct, arranged within the lumen of the conduit, configured to alter the blood flow through the lumen to restrict blood flow in the aorta or vena cava and alter blood flow into one or both kidneys of the patient.

According to another example (“Example 41”), further to the method of Example 37, the indwelling medical device includes an upper portion configured to oppose against a vessel wall of the aorta or vena cava; a lower portion configured to oppose against a vessel wall of the aorta or vena cava; a conduit, arranged between the upper portion and the lower portion, including a lumen for the blood flow through the aorta or vena cava; and the catheter is configured to rotate at least one of the upper portion and the lower portion to alter the blood flow through the lumen to restrict blood flow in the aorta or vena cava and alter blood flow into one or both kidneys.

According to another example (“Example 42”), further to the method of Example 37, the indwelling medical device includes a vessel opposing member portion, arranged at or extending from a distal end of the catheter, including a lumen for the blood flow through the aorta or vena cava, and at least one magnet configured to alter a diameter of the lumen to direct the flow of blood that is pulsating through the aorta or vena cava.

According to another example (“Example 43”), further to the method of Example 37, the indwelling medical device includes a vessel opposing member, arranged at or extending from a distal end of the catheter, including a lumen for the blood flow through the aorta or vena cava, and a cinching band is configured to restrict or constrict a diameter of the vessel opposing member in response to activation of the cinching band.

According to another example (“Example 44”), further to the method of Example 37, the indwelling medical device includes a vessel opposing member, arranged at or extending from a distal end of the catheter, including a lumen for the blood flow through the aorta or vena cava, and a cinching band including an activatable substance that is configured to restrict or constrict a diameter of the vessel opposing member in response to activation of the activatable substance.

According to another example (“Example 45”), further to the method of Example 44, the activatable substance is an electro-active polymer.

According to another example (“Example 46”), further to the method of Example 37, the indwelling medical device includes a vessel opposing member, arranged at or extending from a distal end of the catheter, including a lumen for the blood flow through the aorta or vena cava, and an actuator that is configured to restrict or constrict a diameter of the vessel opposing member in response to activation of the actuator.

According to another example (“Example 47”), further to the method of Example 37, the indwelling medical device includes a vessel opposing member, arranged at or extending from a distal end of the catheter, including a lumen for the blood flow through the aorta or vena cava, and a balloon configured to create a funnel shape at a leading end of the vessel opposing member portion.

According to another example (“Example 48”), further to the method of Example 47, the balloon is configured to draws portions of the vessel opposing member portion inwardly to restrict the lumen in response to inflation.

According to another example (“Example 49”), further to the method of any one of Examples 37-48, the restricting blood flow includes adjusting an amount of restriction based on a patient's activity level.

According to one example (“Example 50”), a device for restricting blood flow within a vessel includes an indwelling medical device coupled to a catheter configured to implant within the vessel configured to restrict blood flow through the indwelling medical device to induce a physiologically mediated therapeutic response.

According to another example (“Example 51”), further to the device of Example 50, the vessel is the aorta or vena cava, and the indwelling medical device includes a vessel opposing member portion arranged at or extending from a distal end of the catheter and configured to oppose against a vessel wall of the aorta or vena cava and a flow altering element, arranged within the vessel opposing member portion and including a lumen for the blood flow through the aorta or vena cava therethrough, configured to alter the blood flow through the lumen to restrict blood flow in the aorta or vena cava and alter blood flow into one or both kidneys of the patient.

According to another example (“Example 52”), further to the device of Example 50, the vessel is the aorta or vena cava, and the indwelling medical device includes a conduit, arranged at or extending from a distal end of the catheter, including a lumen for the blood flow through the aorta or vena cava; and at least one constraining fiber, arranged about a circumference of the conduit, configured to alter the blood flow through the lumen to restrict blood flow in the aorta or vena cava and alter blood flow into one or both kidneys of the patient in response to tension.

According to another example (“Example 53”), further to the device of Example 50, the vessel is the aorta or vena cava, and the indwelling medical device includes a conduit, arranged at or extending from a distal end of the catheter, including a lumen for the blood flow through the aorta or vena cava; and a leaflet construct, arranged within the lumen of the conduit, configured to alter the blood flow through the lumen to restrict blood flow in the aorta or vena cava and alter blood flow into one or both kidneys of the patient.

According to another example (“Example 54”), further to the device of Example 50, the vessel is the aorta or vena cava, and the indwelling medical device includes an upper portion configured to oppose against a vessel wall of the aorta or vena cava; a lower portion configured to oppose against a vessel wall of the aorta or vena cava; a conduit, arranged between the upper portion and the lower portion, including a lumen for the blood flow through the aorta or vena cava; and the catheter is configured to rotate at least one of the upper portion and the lower portion to alter the blood flow through the lumen to restrict blood flow in the aorta or vena cava and alter blood flow into one or

According to another example (“Example 55”), further to the device of Example 50, the vessel is the aorta or vena cava, and the indwelling medical device includes a vessel opposing member portion, arranged at or extending from a distal end of the catheter, including a lumen for the blood flow through the aorta or vena cava, and at least one magnet configured to alter a diameter of the lumen to direct the flow of blood that is pulsating through the aorta or vena cava.

According to another example (“Example 56”), further to the device of Example 50, the vessel is the aorta or vena cava, and the indwelling medical device includes a vessel opposing member, arranged at or extending from a distal end of the catheter, including a lumen for the blood flow through the aorta or vena cava, and a cinching band is configured to restrict or constrict a diameter of the vessel opposing member in response to activation of the cinching band.

According to another example (“Example 57”), further to the device of Example 50, the vessel is the aorta or vena cava, and the indwelling medical device includes a vessel opposing member, arranged at or extending from a distal end of the catheter, including a lumen for the blood flow through the aorta or vena cava, and a cinching band including an activatable substance that is configured to restrict or constrict a diameter of the vessel opposing member in response to activation of the activatable substance.

According to another example (“Example 58”), further to the device of Example 50, the vessel is the aorta or vena cava, and the indwelling medical device includes a vessel opposing member, arranged at or extending from a distal end of the catheter, including a lumen for the blood flow through the aorta or vena cava, and an actuator that is configured to restrict or constrict a diameter of the vessel opposing member in response to activation of the actuator.

According to another example (“Example 59”), further to the device of Example 50, the vessel is the aorta or vena cava, and the indwelling medical device includes a vessel opposing member, arranged at or extending from a distal end of the catheter, including a lumen for the blood flow through the aorta or vena cava, and a balloon configured to create a funnel shape at a leading end of the vessel opposing member portion.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example indwelling flow restriction device, in accordance with various aspects of the present disclosure.

FIG. 2A is a side view of an example indwelling flow restriction device having a flow altering element, in accordance with various aspects of the present disclosure.

FIG. 2B is top down view of the flow altering element, shown in FIG. 2A, in accordance with various aspects of the present disclosure.

FIG. 3A is a side view of another example indwelling flow restriction device having a flow altering element, in accordance with various aspects of the present disclosure.

FIG. 3B is top down view of the flow altering element, shown in FIG. 3A, in accordance with various aspects of the present disclosure.

FIGS. 4A-4F are side views of example lumens for indwelling flow restriction devices, in accordance with various aspects of the present disclosure.

FIG. 5 is a side view of another example indwelling flow restriction device having a flow altering element, in accordance with various aspects of the present disclosure.

FIG. 6 is an illustration of the indwelling flow restriction device shown in FIGS. 3A-B arranged within an aorta of a patient, in accordance with various aspects of the present disclosure.

FIG. 7 is an illustration of the indwelling flow restriction device shown in FIGS. 3A-B arranged within a vena cava of a patient, in accordance with various aspects of the present disclosure.

FIG. 8A is a side view of another example indwelling flow restriction device having a constraining fiber in a first configuration, in accordance with various aspects of the present disclosure.

FIG. 8B is a side view of the indwelling flow restriction device and constraining fiber, shown in FIG. 8A, in a second configuration, in accordance with various aspects of the present disclosure.

FIG. 8C is a side view of the constraining fiber, shown in FIGS. 8A-B, in accordance with various aspects of the present disclosure.

FIG. 9A is a side view of another example indwelling flow restriction device having multiple constraining fibers in a first configuration, in accordance with various aspects of the present disclosure.

FIG. 9B is a side view of the indwelling flow restriction device and constraining fibers, shown in FIG. 9A, in a second configuration, in accordance with various aspects of the present disclosure.

FIG. 10 is an illustration of the indwelling flow restriction device shown in FIGS. 8-9 arranged within a vena cava of a patient, in accordance with various aspects of the present disclosure.

FIG. 11 is an illustration of the indwelling flow restriction device shown in FIGS. 8-9 arranged within an aorta of a patient, in accordance with various aspects of the present disclosure.

FIG. 12A is a side view and top view of another example indwelling flow restriction device having a leaflet construct, in accordance with various aspects of the present disclosure.

FIG. 12B is top down view of the indwelling flow restriction device and the leaflet construct, shown in FIG. 12A, in accordance with various aspects of the present disclosure.

FIGS. 12C-12E are side and top views of the indwelling flow restriction device, shown in FIG. 12A-12B, with different leaflet construct arrangements, in accordance with various aspects of the present disclosure.

FIG. 12F is a partial side view of the indwelling flow restriction device and the leaflet construct, shown in FIGS. 12A-12E, when the leaflet construct is closed, in accordance with various aspects of the present disclosure.

FIG. 12G is a partial side view of the indwelling flow restriction device and the leaflet construct, shown in FIGS. 12A-12F, when the leaflet construct is opened, in accordance with various aspects of the present disclosure.

FIG. 12H is side view of the indwelling flow restriction device and the leaflet construct, shown in FIGS. 12A-12G, in response to venous flow and in accordance with various aspects of the present disclosure.

FIG. 12I is side view of the indwelling flow restriction device and the leaflet construct, shown in FIGS. 12A-12G, in response to arterial flow and in accordance with various aspects of the present disclosure.

FIG. 13 is an illustration of the indwelling flow restriction device shown in FIGS. 12A-I arranged within a vena cava of a patient, in accordance with various aspects of the present disclosure.

FIG. 14 is an illustration of the indwelling flow restriction device shown in FIGS. 12A-I arranged within an aorta of a patient, in accordance with various aspects of the present disclosure.

FIG. 15A is a side view of another example indwelling flow restriction device in a first configuration, in accordance with various aspects of the present disclosure.

FIG. 15B is a side view of the indwelling flow restriction device, shown in FIG. 15A, in a second configuration, in accordance with various aspects of the present disclosure.

FIG. 16A are top and cross-sectional views of the indwelling flow restriction device, shown in FIGS. 15A-15B, in the first configuration, in accordance with various aspects of the present disclosure.

FIG. 16B is a side view of the indwelling flow restriction device, shown in FIGS. 15A-B, in the second configuration, in accordance with various aspects of the present disclosure.

FIG. 17 is a side view of the indwelling flow restriction device, shown in FIGS. 15A-15B and FIGS. 16A-16B, in accordance with various aspects of the present disclosure.

FIG. 18 is an illustration of the indwelling flow restriction device shown in FIGS. 15A-17 in a first configuration arranged within a vena cava of a patient, in accordance with various aspects of the present disclosure.

FIG. 19 is an illustration of the indwelling flow restriction device shown in FIGS. 15A-17 in a second configuration arranged within a vena cava of a patient, in accordance with various aspects of the present disclosure.

FIG. 20 is an illustration of the indwelling flow restriction device shown in FIGS. 15A-17 in a first configuration arranged within an aorta of a patient, in accordance with various aspects of the present disclosure.

FIG. 21 is an illustration of the indwelling flow restriction device shown in FIGS. 15A-17 in a second configuration arranged within an aorta of a patient, in accordance with various aspects of the present disclosure.

FIG. 22 is a side view of another example indwelling flow restriction device having a flow altering element, in accordance with various aspects of the present disclosure.

FIG. 23A is a side view of another example indwelling flow restriction device in a first configuration, in accordance with various aspects of the present disclosure.

FIG. 23B is a side view of the indwelling flow restriction device, shown in FIG. 23A, in a second configuration, in accordance with various aspects of the present disclosure.

FIG. 24A is a side view of another example indwelling flow restriction device in a first configuration, in accordance with various aspects of the present disclosure.

FIG. 24B is a side view of the indwelling flow restriction device, shown in FIG. 24A, in a second configuration, in accordance with various aspects of the present disclosure.

FIG. 25A is a side view of another example indwelling flow restriction device in a first configuration, in accordance with various aspects of the present disclosure.

FIG. 25B is a side view of the indwelling flow restriction device, shown in FIG. 25A, in a second configuration, in accordance with various aspects of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Various aspects of the present disclosure are directed toward methods that include arranging an indwelling flow restriction device within the aorta or vena cava of a patient. The methods may also include adjusting the indwelling flow restriction device to redirect some portion of blood flow into at least one branch vessel of the aorta while maintaining a substantially unrestricted blood flow within the aorta proximal to the branch vessel. In the case of use in the vena cava, methods may also include adjusting the indwelling flow restriction device to increase blood flow from at least one tributary vessel of the vena cava.

In addition, the indwelling flow restriction devices, as discussed in further detail below, may be configured to indwell within an aorta or vena cava of a patient. In instances where an indwelling flow restriction device is arranged within the aorta, the device may also be configured to alter blood flow into at least one branch vessel of the aorta while maintaining a substantially unrestricted blood flow within the aorta proximal to the branch vessel. In instances where an indwelling flow restriction device is arranged within the vena cava, the device may also be configured to increase blood flow from at least one tributary vessel of the vena cava.

When implanted in the aorta, the indwelling flow restriction devices are configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the indwelling flow restriction devices are configured to create a narrowed flow lumen in the conduit located in the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the indwelling flow restriction device may augment perfusion from a tributary vessel (e.g., renal veins) terminating in the vena cava by altering pressure within the vena cava to alter blood flow from the tributary vessel of the vena cava. In certain instances, the indwelling flow restriction device 100 may be configured to create a narrowed flow lumen in the conduit located in the vena cava distal of the at least one tributary vessel of between about 40% and about 90%. Use of the indwelling flow restriction devices, discussed in further detail below, by dropping pressure in the renal veins may increase kidney perfusion hemodynamically rather than pharmaceutically.

In certain instances, the flow restriction devices discussed herein may be implanted in other vessels. The flow restriction devices may facilitate increase in peripheral resistance to treat decreases in blood pressure or resistance within the vasculature. As discussed further below, this may include implantation of the flow restriction devices for treatment of an arteriovenous (AV) fistula.

The indwelling flow restriction devices, as discussed in further detail below, are directed toward treating symptoms of congestive heart failure in a patient and/or other cardiovascular diseases such as hypertension and hypotension or other medical conditions or procedures that may result in impaired kidney perfusion that could lead to Acute Kidney Injury (AKI). In certain instances, patients with heart failure (such as late-stage heart failure) have decreased cardiac output (e.g., amount of blood pumped by the heart per minute), which can lead to decreased diuresis. The indwelling flow restriction devices, in certain instances, increase natural diuresis and lessen buildup of excess fluid that results from heart failure.

The indwelling flow restriction device may be used for acute treatment with a heart failure patient under physician's care (e.g., hospitalized). The indwelling flow restriction devices may be configured to induce a physiologically mediated therapeutic response by reducing buildup of excess fluid (e.g., hypervolemia) in the body, as the buildup of fluid in combination with an already failing heart may further harm the patient. As discussed in further detail below, various aspects of the present discourse are directed toward mitigating against buildup of excess fluid and diverting excess fluid from the heart for acute treatment.

The indwelling flow restriction devices may be implanted for hours, and/or days in an acute and monitored setting. The amount or resistance or flow restriction applied by the indwelling flow restriction device may be adjusted after implantation to meet patient needs. Clinically, measurement of ankle pressure, Doppler ultrasound velocity, ankle-brachial index, or other hemodynamic parameters in the lower limbs can be employed to optimize the magnitude of the induced stenosis while ensuring adequate limb perfusion.

The indwelling flow restriction devices may reduce activation of a sympathetic nervous system of the patient to decrease resting heart rate, blood pressure, and/or N-terminal pro b-type natriuretic peptide (nt-proBNP). In addition, reducing activation of the sympathetic nervous system may also improve heart contractility. In certain instances, the indwelling flow restriction devices reduces stimulation of a Renin-Angiotensin-Aldosterone system (RAAS) of the patient.

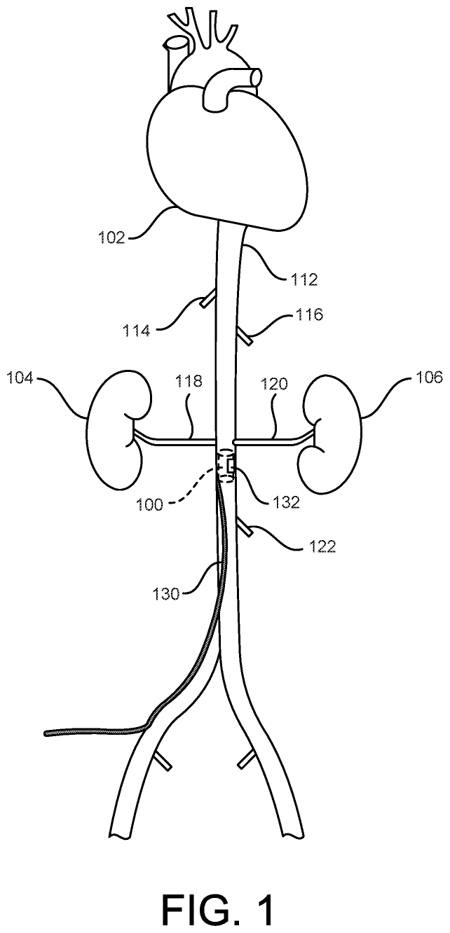

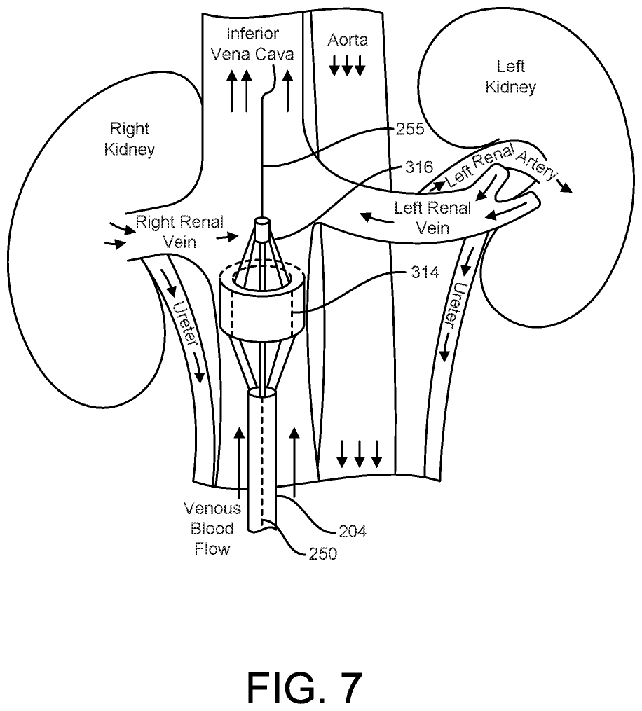

FIG. 1 shows an example indwelling flow restriction device 100 in accordance with various aspects of the present disclosure. The indwelling flow restriction device 100 is shown arranged within a patient's vasculature. A simplified diagram of the patient's vasculature shown in FIG. 1 includes the patient's heart 102, kidneys 104, 106, aorta 112, or vena cava 112, celiac artery 114, superior mesenteric artery 116, vessels 118, 120 that supply the kidneys 104, 106 (renal arteries) or drain the kidneys 104, 106 (renal veins), and inferior mesenteric artery 122. The indwelling flow restriction device 100 may be arranged within the aorta 112. As shown in FIGS. 7, 10, 13, and 16-18, the indwelling flow restriction device 100 may also be placed in the patient's' vena cava. In certain instances, the indwelling flow restriction device 100 is arranged within the aorta 112 distal of the vessels 118, 120 that supply the kidneys 104, 106 (renal arteries) or drain the kidneys 104, 106 (renal veins).

The indwelling flow restriction device 100 may be coupled to a catheter 130 that facilitates placement, arrangement, and/or retrieval of the indwelling flow restriction device 100 within the aorta 112. In certain instances, the catheter 130 may include features, as discussed in further detail below, that facilitate diametric adjustment (or other structural change) of the indwelling flow restriction device 100. Changing dimensions of the indwelling flow restriction device 100 may increase or decrease resistance to blood flow through the indwelling flow restriction device 100 to increase blood flow into one or both of the kidneys 104, 106.

As noted above, the implantable flow restriction devices 100, discussed herein, may include a sensor 132. The sensor 132 may be configured to monitor blood flow (or other hemodynamic parameters) in or near the implantable flow restriction device 100. The sensor 132 may, alternatively, monitor various biochemical, biomarker, or pharmacological parameters in the blood stream. The sensor 132 may be one of a pressure sensor or a flow sensor. In addition, the implantable flow restriction device 100 may be configured to alter the amount of blood flow in response to the blood flow monitored by the sensor 132. The amount of restriction provided by the implantable flow restriction device 100 may be adjusted to achieve a desired blood flow into the kidneys 104, 106. The sensor 132 may be used in combination with sources of information external to the patient to assist in the amount of pressure applied by the implantable flow restriction device 100 to achieve a desired blood flow into one or both of the kidneys 104, 106. In certain instances, the sensor 132 may be utilized to employ feedback loops to set an amount of restriction of the implantable flow restriction device 100. The sensor 132 may be set to monitor pre-determined physiological or other inputs in order to control, for example, length of duty cycle (on/off of restriction), speed of duty cycle, temporary suspension, and/or shut off of treatment to ensure patient safety depending on physiological monitoring. The sensor 132 may also utilize physician or patient to control when restriction is active.

The illustrative indwelling flow restriction device 100 shown in FIG. 1 is not intended to suggest any limitation as to the scope of use or functionality of embodiments of the disclosure disclosed throughout this document. Neither should the illustrative indwelling flow restriction device 100 be interpreted as having any dependency or requirement related to any single component or combination of components illustrated therein. Additionally, any one or more of the components depicted in FIG. 1 can be, in embodiments, integrated with various ones of the other components depicted therein (and/or components not illustrated). For example, the indwelling flow restriction device 100 discussed below may include the sensor 132 arranged within or on an outer surface of the indwelling flow restriction device 100. In addition, the flow restriction device 100 may be implanted in other vessels. The flow restriction device 100 may facilitate increase in peripheral resistance to treat decreases in blood pressure or resistance within the vasculature. This may include implantation of the flow restriction devices for treatment of an arteriovenous (AV) fistula.

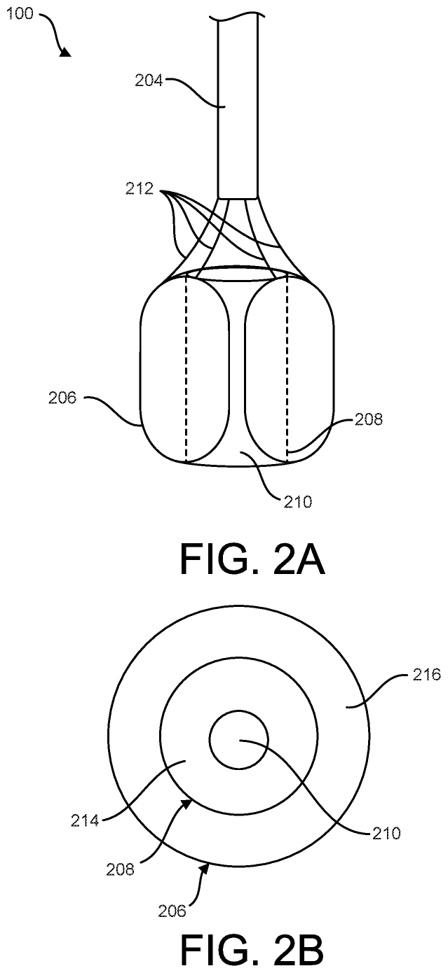

FIG. 2A is a side view of an example indwelling flow restriction device 100 having a flow altering element 208, in accordance with various aspects of the present disclosure. The indwelling medical device 100, as shown in FIGS. 2A-B, may be used for acute alteration of blood flow in an aorta or vena cava of a patient. The indwelling medical device 100 may also include a catheter 204, and a vessel opposing member portion arranged at or extending from a distal end of the catheter 204. In certain instances and as shown, the flow altering element 208 may be a balloon. In other instances, the flow altering element 208 may be a stent, stent-graft, graft, valve and flap device, restriction band, or other similar devices. The vessel opposing member portion is configured to oppose against a vessel wall of the aorta or vena cava. In certain instances and as shown, the vessel opposing member portion is an outer balloon 206. The outer balloon 206 is configured to inflate and deflate to oppose against a vessel wall of the aorta or vena cava.

In certain instances, the flow altering element 208 is arranged within the outer balloon 206 (e.g., the vessel opposing member portion). The flow altering element 208 also includes a lumen 210 for directing the flow of blood that is pulsating through the aorta or vena cava. The flow altering element 208 may be configured to alter the blood flow through the lumen 210 to restrict blood flow in the aorta or vena cava and alter blood flow into or out of one or both kidneys of the patient. In certain instances, the flow altering element 208 is configured to inflate and deflate to alter a level of restriction of blood flow through the lumen 210. The outer balloon 206 may contact the vessel wall of the aorta or vena cava such that the blood flow through the aorta or vena cava, at the location the indwelling flow restriction device 100, is forced solely through the lumen 210. Thus, the flow altering element 208 is configured to alter the amount of restriction of blood flow through the aorta or vena cava at the location of the indwelling flow restriction device 100. In certain instances and as shown, the outer balloon 206 and the flow altering element 208 are of substantially similar lengths.

In certain instances, the outer balloon 206 is configured to inflate to secure or oppose the indwelling flow restriction device 100 at a desired location within the aorta or vena cava or another vessel. After the indwelling flow restriction device 100 is secured or opposed, the flow altering element 208 may be selectively inflated and deflated to alter an amount of restriction of the lumen 210. The flow altering element 208 may be configured to inflate and deflate to selected or achieve a desired level of restriction of the aorta, vena cava, or other vessel. The flow altering element 208 and the outer balloon 206 may be connected to one or more of a plurality of inflation ports 212 arranged through the catheter 204.

The inflation ports 212 may be coupled to a control system that inflates and deflates the flow altering element 208. The control system may cyclically inflate and deflate the flow altering element 208 to maximize therapeutic effect and minimize potential safety risks. The flow altering element 208 pressure may be checked periodically (e.g., every 15 minutes), and the amount of flow restriction may include periods of little or no restriction to meet oxygen demand that may be underserved during periods of higher flow redirection. A physiological indicator, such as pedal pulse, may be checked periodically (e.g., every 15 minutes) to assess the effect of the therapy on peripheral perfusion.

FIG. 2B is top down view of the flow altering element 208, shown in FIG. 2A, in accordance with various aspects of the present disclosure. As shown in FIG. 2B, the outer balloon 206 includes a reservoir 216 and the flow altering element 208 includes a reservoir 214. Each of the reservoirs 214, 216 may be separated by being inflated and deflated with gas, liquid, or another suitable substance to maintain a desired size of the flow altering element 208 and the outer balloon 206.

In certain instances, the outer balloon 206 is configured to oppose the vessel wall in the aorta and force primary blood flow through the central lumen of the flow altering element 208. The flow altering element 208 can be inflated or deflated to create a central flow lumen distal of one or both renal arteries, between about 40% and about 80% the diameter of the natural aorta to alter blood flow into the at least one branch vessel of the aorta. In instances where the flow altering element 208 is not balloon, a diameter of the flow lumen of the flow altering element 208 may be altered to adjust restriction. In other instances, the outer balloon 206 is configured to oppose against the vessel wall in the vena cava to create a narrowed flow lumen in the conduit located in the vena cava distal of one or both renal veins of between about 40% and about 90% to alter blood flow through one or both of the renal veins. The outer balloon 206 may be arranged upstream from one or both of the renal veins and the flow altering element 208 may be configured to drop pressure blood out of one or both of the renal veins to pull blood flow through the renal arteries.

In certain instances, the flow altering element 208 is arranged in a vessel other than the aorta or venal cava as noted above. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

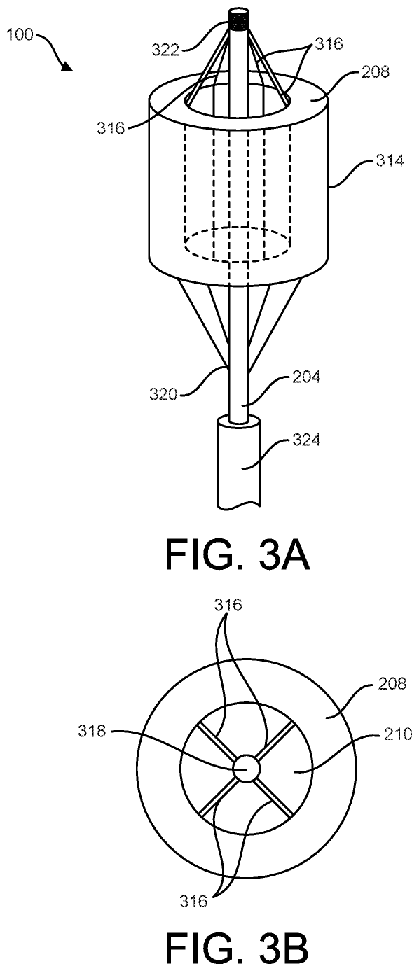

FIG. 3A is a side view of another example indwelling flow restriction device 100 having a flow altering element 208, in accordance with various aspects of the present disclosure. The indwelling medical device 100, as shown in FIGS. 3A-B, may be used for acute alteration of blood flow in an aorta or vena cava of a patient. The indwelling medical device 100 may also include a catheter 204, and the vessel opposing member portion 314 arranged at or extending from a distal end of the catheter 204. The vessel opposing member portion 314 is configured to oppose against a vessel wall of the aorta or vena cava. In certain instances and as shown, the vessel opposing member portion 314 may be a stent, a balloon, a polymer material, or another support material. The vessel opposing member portion 314 is configured to oppose against a vessel wall of the aorta or vena cava. The vessel opposing member portion 314 may include a stent frame arranged along a length of the flow altering element 208. The flow altering element 208 may be a balloon, stent, stent-graft, restricting band, or other similar device.

In certain instances, the flow altering element 208 is arranged within the vessel opposing member portion 314. The vessel opposing member portion 314 may contact the vessel wall of the aorta or vena cava such that the blood flow through the aorta or vena cava, at the location the indwelling flow restriction device 100, is forced solely through the lumen 210.

The flow altering element 208 also includes a lumen 210 for directing the flow of blood that is pulsating through the aorta or vena cava. The flow altering element 208 may be configured to alter the blood flow through the lumen 210 to restrict blood flow in the aorta or vena cava and alter blood flow into one or both kidneys of the patient. In certain instances, the flow altering element 208, configured as balloon or inflatable sheath, is configured to inflate and deflate to alter a level of restriction of blood flow through the lumen 210. In instances where the flow altering element 208 is not balloon, a diameter of the flow lumen of the flow altering element 208 may be altered to adjust restriction. Thus, the flow altering element 208 is configured to alter the amount of restriction of blood flow through the aorta or vena cava at the location of the indwelling flow restriction device 100.

In certain instances, a plurality of struts 316 may be coupled to the catheter 204 and arranged within the vessel opposing member portion 314. As shown in FIG. 3A, the plurality of struts 316 extend outwardly from a proximal location 320 along the catheter 204, through the lumen 210 of the flow altering element 208, and extend inwardly after exiting the lumen 210 toward a distal end 322 of the catheter 204. The plurality of struts 316 may be configured to support the flow altering element 208 and/or the vessel opposing member portion 314.

The indwelling flow restriction device 100 may also include a delivery sheath 324. The flow altering element 208 and the vessel opposing member portion 314 may be configured to collapse to a delivery diameter within the delivery sheath 324 and deploy against the vessel wall to a deployed diameter as shown in FIG. 3A. The vessel opposing member portion 314 may include a self-expanding stent or the vessel opposing member portion 314 may expand against the vessel wall and secure to or oppose against the vessel wall in response to inflation of the flow altering element 208. In addition, the vessel opposing member portion 314 may include a graft portion.

In certain instances, the vessel opposing member portion 314 may include expandable portions or the entire vessel opposing member portion 314 may be balloon expandable. For example, expandable portions of the vessel opposing member portion 314 may be configured to have controlled expansion that allows diametric adjustment beyond an initial deployment diameter through a plurality of adjusted diameters up to a maximum diametric expansion limit of the vessel opposing member portion 314. Examples of diametrically adjustable devices and associated methods are also described in U.S. Patent Publication 2016/0143759.

Examples of suitable stent patterns and associated methods of manufacture are also described in U.S. Pat. No. 6,673,102. Stents of the vessel opposing member portion 314 may be formed from a variety of wire materials, including stainless steel, nickel-titanium alloy (nitinol), tantalum, elgiloy, various polymer materials, such as poly(ethylene terephthalate) (PET) or polytetrafluoroethylene (PTFE), or bioresorbable materials, such as levorotatory polylactic acid (L-PLA) or polyglycolic acid (PGA).

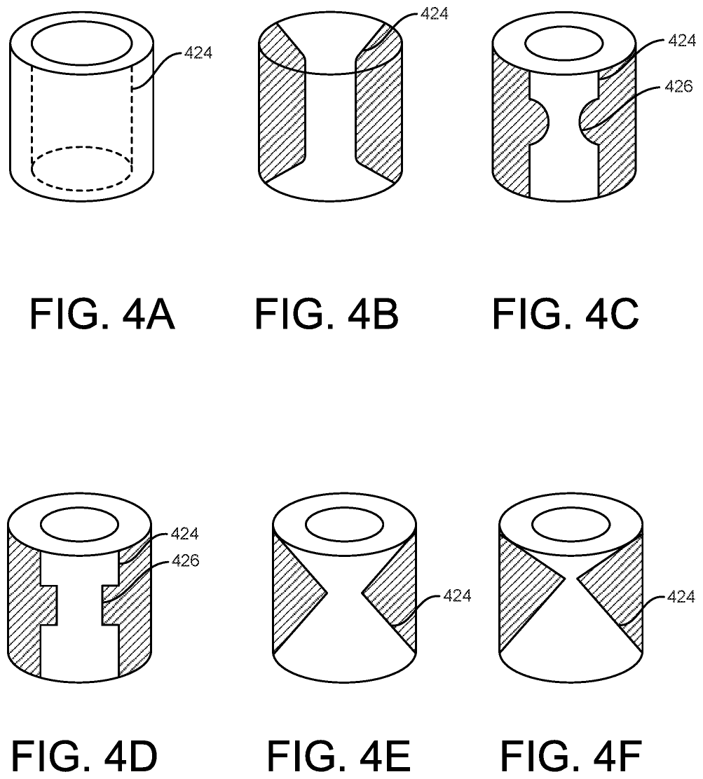

FIG. 3B is top down view of the flow altering element 208, shown in FIG. 3A, in accordance with various aspects of the present disclosure. As shown, the plurality of struts 316 divide the lumen 210. The catheter 204 may also include a guidewire lumen 318 to facilitate delivery. As shown, the flow altering element 208 has an internal shape that is substantially cylindrical. In certain instances and as shown in FIGS. 4A-F, the flow altering element 208, and therefore the lumen 210 of the flow altering element 208, may have an internal hourglass shape, a shape with circular restriction portions, a shape with stepped restriction portions, a ramped shape, and a peaked shape. The flow altering element 208 may form these shapes in a restricted configuration and include a nominal, non-restricted, shape or a cylinder. In certain instances, the flow altering element 208 includes these shapes in either a restricted or non-restricted configuration.

In certain instances, the flow altering element 208 is arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

FIGS. 4A-F are side views of example lumens for indwelling flow restriction devices 100, in accordance with various aspects of the present disclosure. The shapes shown in FIGS. 4A-F may be formed in internal surfaces of the indwelling flow restriction devices 100 or in internal surfaces of flow altering elements 208 discussed herein.

As shown in FIG. 4A, the internal surfaces 424 of the indwelling flow restriction devices 100 or in internal surfaces 424 of flow altering elements 208 may be substantially cylindrical. As shown in FIG. 4B, the internal surfaces 424 of the indwelling flow restriction devices 100 or in internal surfaces 424 of flow altering elements 208 may be of an hour glass shape. As shown in FIG. 4C, the internal surfaces 424 of the indwelling flow restriction devices 100 or in internal surfaces 424 of flow altering elements 208 may include circular or semi-circular indentations 426. As shown in FIG. 4D, the internal surfaces 424 of the indwelling flow restriction devices 100 or in internal surfaces 424 of flow altering elements 208 may include stepped indentations 426. As shown in FIG. 4E, the internal surfaces 424 of the indwelling flow restriction devices 100 or in internal surfaces 424 of flow altering elements 208 may include v-shaped indentations 426. As shown in FIG. 4F, the internal surfaces 424 of the indwelling flow restriction devices 100 or in internal surfaces 424 of flow altering elements 208 may form a ramped structure.

The internal surfaces 424 of the indwelling flow restriction devices 100 or in internal surfaces 424 shown in FIGS. 4A-F may be consistent over the entire circumference (360 degrees) of the internal surfaces 424 of the indwelling flow restriction devices 100 or in internal surfaces 424. In other instances, the internal surfaces 424 of the indwelling flow restriction devices 100 or in internal surfaces 424 shown in FIGS. 4A-F may be formed in portions or sections (e.g., pockets spaced 180 degrees) of the internal surfaces 424 of the indwelling flow restriction devices 100 or in internal surfaces 424.

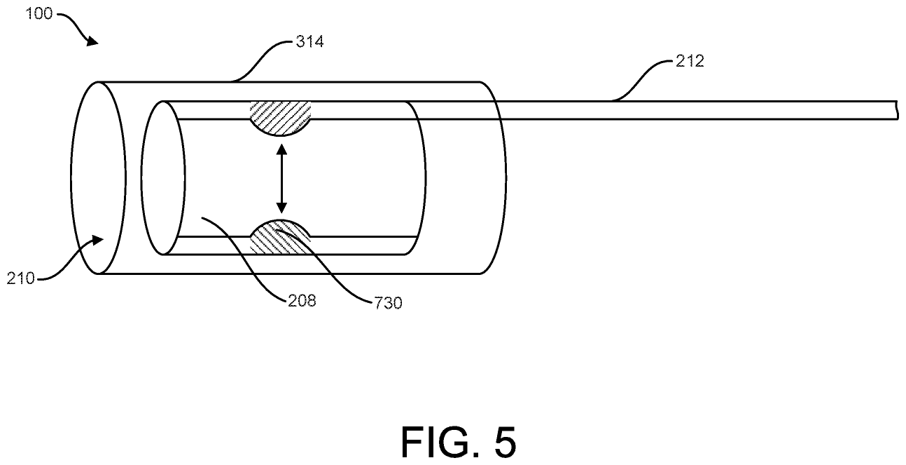

FIG. 5 is a side view of another example indwelling flow restriction device having a flow altering element, in accordance with various aspects of the present disclosure. The indwelling medical device 100, as shown in FIG. 5, is a more detailed view of the shape shown in FIG. 4C. The device 100 may be used for acute alteration of blood flow in an aorta or vena cava of a patient. The indwelling medical device 100 may also include a catheter (not shown), and a vessel opposing member portion 314 arranged at or extending from a distal end of the catheter. The vessel opposing member portion 314 is configured to oppose against a vessel wall of the aorta or vena cava. In certain instances and as shown, the vessel opposing member portion 314 may be a stent, a balloon, a polymer material, or another support material, in part or whole. The vessel opposing member portion 314 is configured to oppose against a vessel wall of the aorta or vena cava.

In certain instances, the flow altering element 208 is arranged within the vessel opposing member portion 314. The vessel opposing member portion 314 may contact the vessel wall of the aorta or vena cava such that the blood flow through the aorta or vena cava, at the location the indwelling flow restriction device 100, is forced solely through the lumen 210. The flow altering element 208 may include a length less than a length of the vessel opposing member portion 314 as shown in FIG. 5.

The flow altering element 208 also includes a lumen 210 for directing the flow of blood that is pulsating through the aorta or vena cava. The flow altering element 208 may be configured to alter the blood flow through the lumen 210 to restrict blood flow in the aorta or vena cava and alter blood flow into one or both kidneys of the patient. In certain instances, the flow altering element 208, as a balloon or inflatable sheath, is configured to inflate and deflate to alter a level of restriction of blood flow through the lumen 210 by supplying and returning air, liquid, or another substance through an inflation port 212. In instances where the flow altering element 208 is not balloon, a diameter of the flow lumen of the flow altering element 208 may be altered to adjust restriction. Thus, the flow altering element 208 is configured to alter the amount of restriction of blood flow through the aorta or vena cava at the location of the indwelling flow restriction device 100.

After the indwelling flow restriction device 100 is secured or opposed, the flow altering element 208 may be selectively inflated and deflated (or restricted and unrestricted) to alter an amount of restriction of the lumen 210. A bladder 730 may be formed by the flow altering element 208 that is consistent over the entire circumference (360 degrees) of the internal surfaces of the flow altering element 208 or formed in portions or sections (e.g., pockets spaced 180 degrees) of the internal surfaces of the flow altering element 208.

As noted above, the indwelling flow restriction device 100 is may be arranged in a patient's aorta, vena cava, or in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

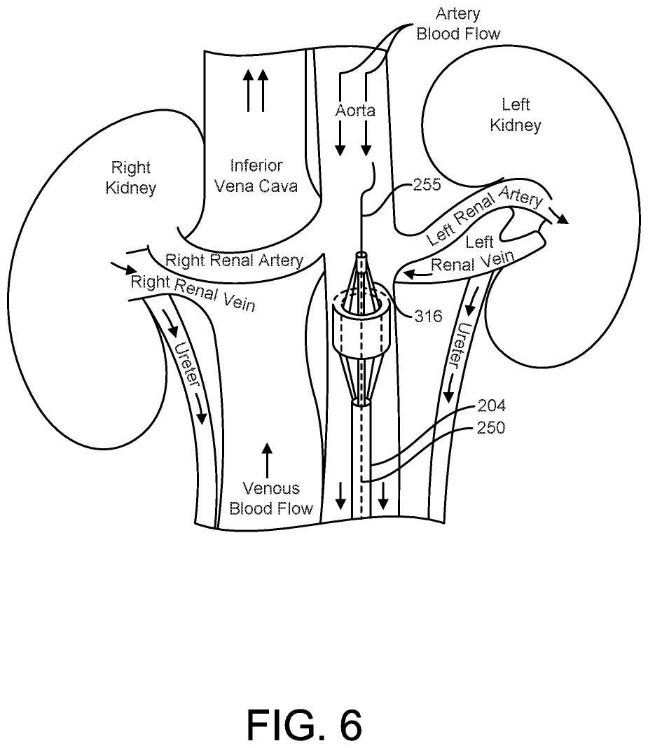

FIG. 6 is an illustration of the indwelling flow restriction device shown in FIGS. 3A-B arranged within an aorta of a patient, in accordance with various aspects of the present disclosure and FIG. 7 is an illustration of the indwelling flow restriction device shown in FIGS. 3A-B arranged within a vena cava of a patient, in accordance with various aspects of the present disclosure. As shown in FIG. 6 and FIG. 7 (and as described in detail above), the catheter 204 may include a guide wire lumen 250 to facilitate arrangement of a guidewire 255 to deliver the device 100 to a target location. The device 100 may be arranged distal of the renal veins or arteries.

When implanted in the aorta, the indwelling flow restriction device 100 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the indwelling flow restriction devices are configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the indwelling flow restriction device 100 may augment perfusion from a tributary vessel (e.g., renal veins) terminating in the vena cava by altering pressure within the vena cava to alter blood flow from the tributary vessel of the vena cava. In certain instances, the indwelling flow restriction device 100 may be configured to create a narrowed flow lumen in the conduit located in the vena cava distal of the at least one tributary vessel of between about 40% and about 90%. Use of the indwelling flow restriction devices, discussed in further detail below, by dropping pressure in the renal veins may increase kidney perfusion hemodynamically rather than pharmaceutically.

As noted above, the indwelling flow restriction device 100 is may be arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

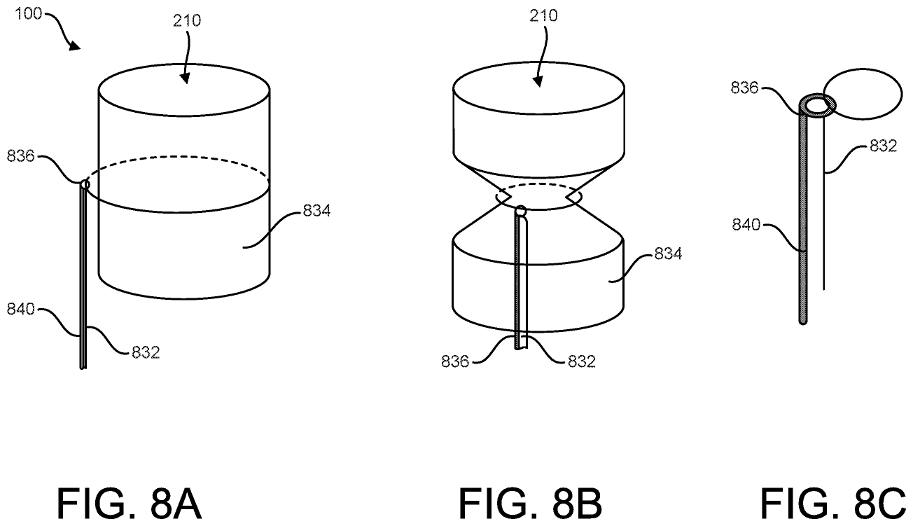

FIG. 8A is a side view of another example indwelling flow restriction device 100 having a constraining fiber 832 in a first configuration, in accordance with various aspects of the present disclosure. The indwelling medical device 100, as shown in FIGS. 8A-C, may be used for acute alteration of blood flow in an aorta or vena cava of a patient. The indwelling medical device 100 may also include an eyelet support member 840, and a conduit 834 arranged at or extending from a distal end of the eyelet support member 840. The conduit 834 includes a lumen 210 for the blood flow through the aorta or vena cava. In certain instances, the conduit 834 includes diametrically adjustable stent and/or graft components. In addition, the conduit 834 is configured to oppose against a vessel wall of the aorta or vena cava.

In certain instances and as shown, the constraining fiber 832 is arranged about a circumference of the conduit 834. The eyelet support member 840 may be configured to maintain a location of the constraining fiber 832 relative to the conduit 834. The constraining fiber 832 is configured to alter the blood flow through the lumen 210 to restrict blood flow in the aorta or vena cava and alter blood flow into one or both kidneys of the patient in response to tension. As shown in FIG. 8A, the constraining fiber 832 is in a first configuration and is not in tension. In this configuration, the conduit 834 is not restricting flow. FIG. 8B is a side view of the indwelling flow restriction device 100 and the constraining fiber 832, shown in FIG. 8A, in a second configuration. In the second configuration, flow is restricted. Tension is applied to the constraining fiber 832 and the conduit 834 is restricted.

The constraining fiber 832 may be configured to alter the blood flow through the lumen 210 to restrict blood flow in the aorta or vena cava and alter blood flow into one or both kidneys of the patient. In certain instances, the constraining fiber 832 is configured to a level of restriction of blood flow through the lumen 210 based on the tension applied. Thus, the constraining fiber 832 is configured to alter the amount of restriction of blood flow through the aorta or vena cava at the location of the indwelling flow restriction device 100. Examples of constraining fibers can be found in U.S. Pat. No. 10,117,765 to Norris et al.

FIG. 8C is a side view of the constraining fiber 832, shown in FIGS. 8A-B, in accordance with various aspects of the present disclosure. The constraining fiber 832 is arranged through an eyelet 836 in the eyelet support member 840. The eyelet 836 may facilitate the application of force about the conduit 834.

In certain instances, the conduit 834 is arranged in a vessel other than the aorta or venal cava. In these instances, the constraining fiber 832 may be configured to alter the blood flow through the lumen 210 to restrict blood flow in the vessel and induce a physiologically mediated therapeutic response in the patient. In certain instances, the constraining fiber 832 is configured to induce the physiologically mediated therapeutic response to include an increase in peripheral resistance within the vessel. The constraining fiber 832 may be configured to treat a fistula within the vessel and increase in peripheral resistance within the vessel as described in further detail below.

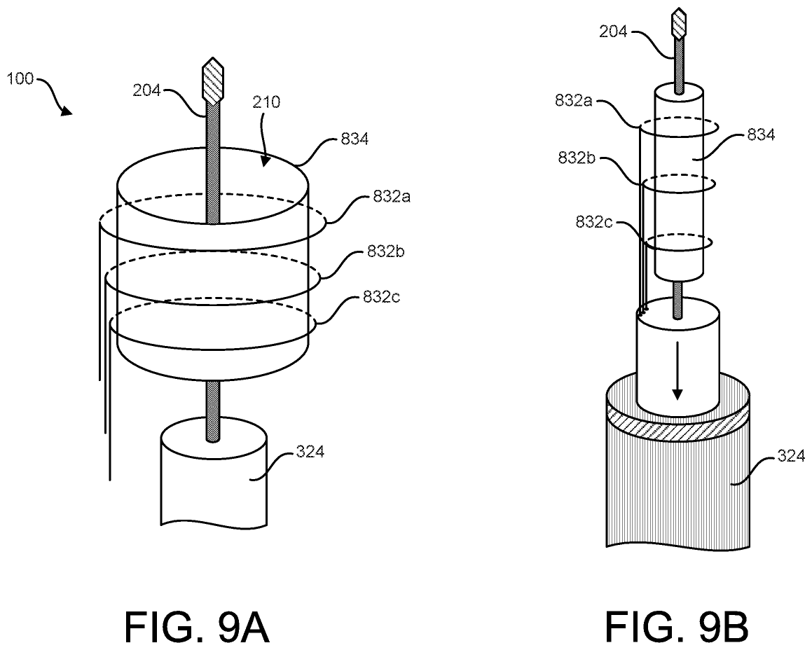

FIG. 9A is a side view of another example indwelling flow restriction device having multiple constraining fibers 832a-c in a first configuration, in accordance with various aspects of the present disclosure. The indwelling medical device 100, as shown in FIGS. 9A-B, may be used for acute alteration of blood flow in an aorta or vena cava of a patient. The indwelling medical device 100 may also include a catheter 204, and a conduit 834 arranged at or extending from a distal end of the catheter 204. The conduit 834 includes a lumen 210 for the blood flow through the aorta or vena cava. In certain instances, the conduit 834 includes diametrically adjustable stent and/or graft components. In addition, the conduit 834 is configured to oppose against a vessel wall of the aorta or vena cava.

In certain instances and as shown, the constraining fibers 832a-c are arranged about a circumference of the conduit 834. The constraining fibers 832a-c are arranged at different locations along a length of the conduit 834. In addition, the constraining fibers 832a-c may be individually tensioned to restrict a diameter of the conduit 834 at the location of the constraining fibers 832a-c. The constraining fibers 832a-c are configured to alter the blood flow through the lumen 210 to restrict blood flow in the aorta or vena cava and alter blood flow into one or both kidneys of the patient in response to tension.

The constraining fibers 832a-c may be configured to alter the blood flow through the lumen 210 to restrict blood flow in the aorta or vena cava and alter blood flow into one or both kidneys of the patient. In certain instances, the constraining fibers 832a-c are configured to a level of restriction of blood flow through the lumen 210 based on the tension applied. Thus, the constraining fibers 832a-c are configured to alter the amount of restriction of blood flow through the aorta or vena cava at the location of the indwelling flow restriction device 100.

As noted above, the indwelling flow restriction device 100 is may be arranged in a vessel other than the aorta or venal cava. In these instances, the constraining fibers 832a-c may be configured to alter the blood flow through the lumen 210 to restrict blood flow in the vessel and induce a physiologically mediated therapeutic response in the patient. In certain instances, the indwelling flow restriction device 100 may be configured to induce the physiologically mediated therapeutic response to include an increase in peripheral resistance within the vessel. The indwelling flow restriction device 100 may be configured to treat a fistula within the vessel and increase in peripheral resistance within the vessel as described in further detail below.

FIG. 9B is a side view of the indwelling flow restriction device 100 and constraining fibers 832a-c, shown in FIG. 9A, in a second configuration, in accordance with various aspects of the present disclosure. The indwelling flow restriction device 100 may also include a delivery sheath 324. The conduit 834 may be configured to collapse to a delivery diameter within the delivery sheath 324 and deploy against the vessel wall to a deployed diameter as shown in FIG. 9A. The conduit 834 may include a self-expanding stent and/or graft and may expand against the vessel wall and secure to or oppose against the vessel wall.

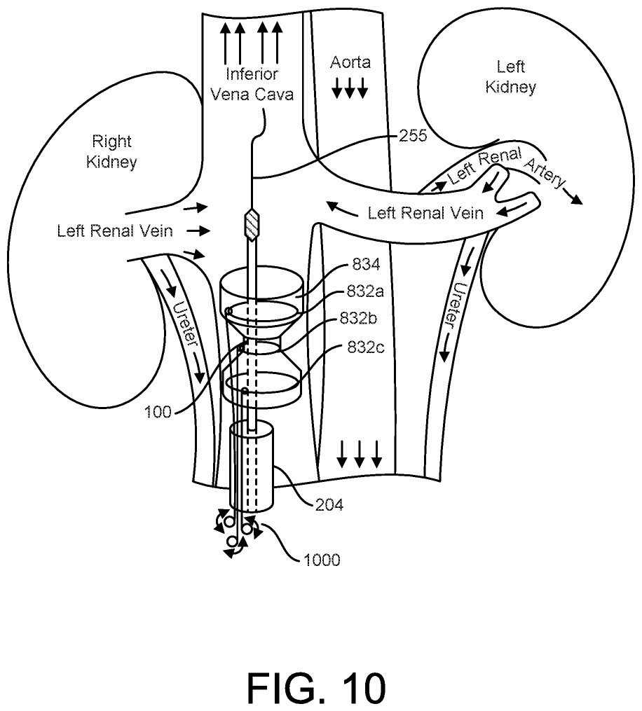

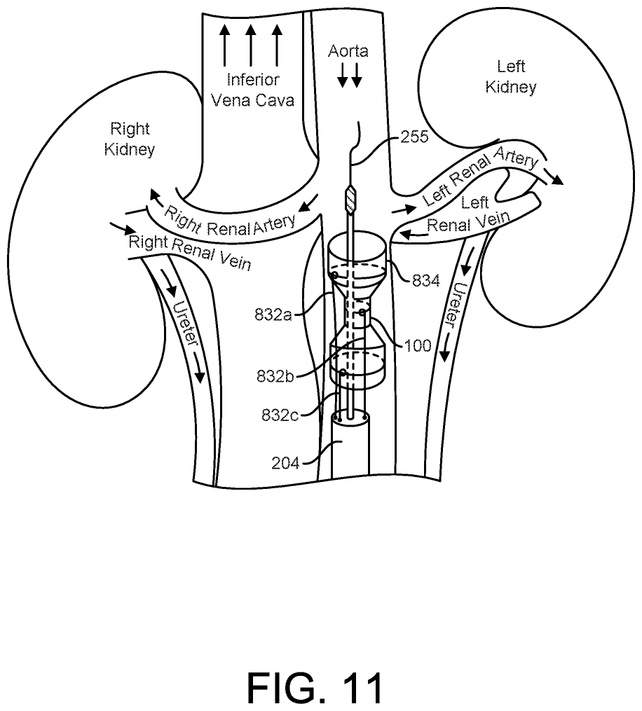

FIG. 10 is an illustration of the indwelling flow restriction device shown in FIGS. 8-9 arranged within a vena cava of a patient, in accordance with various aspects of the present disclosure. FIG. 11 is an illustration of the indwelling flow restriction device shown in FIGS. 8-9 arranged within an aorta of a patient, in accordance with various aspects of the present disclosure. As shown in FIG. 10, the device 100 may be coupled to a catheter handle 1000. The catheter handle 1000 may include elements or structures coupled to constraining fibers 832a-c that are configured to facilitate application of tension to the constraining fibers 832a-c by a user. The other devices 100 discussed herein may also be similarly coupled to a catheter handle 1000 (e.g., actuation members 1244 coupled to the handle 1000 and as described in detail below) to allow a user to open and close the devices. In addition, the catheter handle 1000 may include visual indicators to display the amount of restriction applied.

When implanted in the aorta, the indwelling flow restriction device 100 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the indwelling flow restriction devices are configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the indwelling flow restriction device 100 may augment perfusion from a tributary vessel (e.g., renal veins) terminating in the vena cava by altering pressure within the vena cava to alter blood flow from the tributary vessel of the vena cava. In certain instances, the indwelling flow restriction device 100 may be configured to create a narrowed flow lumen in the conduit located in the vena cava distal of the at least one tributary vessel of between about 40% and about 90%. Use of the indwelling flow restriction devices, discussed in further detail below, by dropping pressure in the renal veins may increase kidney perfusion hemodynamically rather than pharmaceutically.

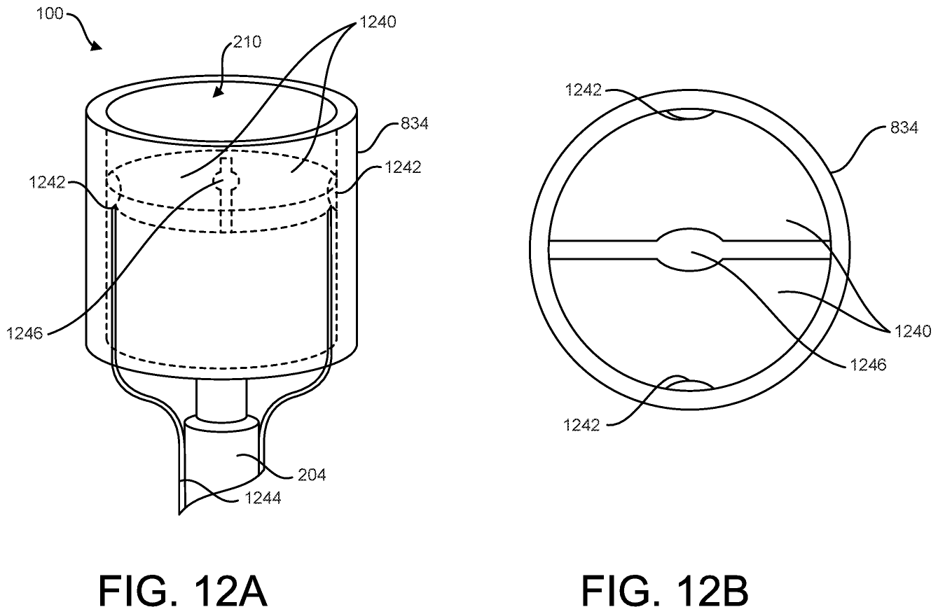

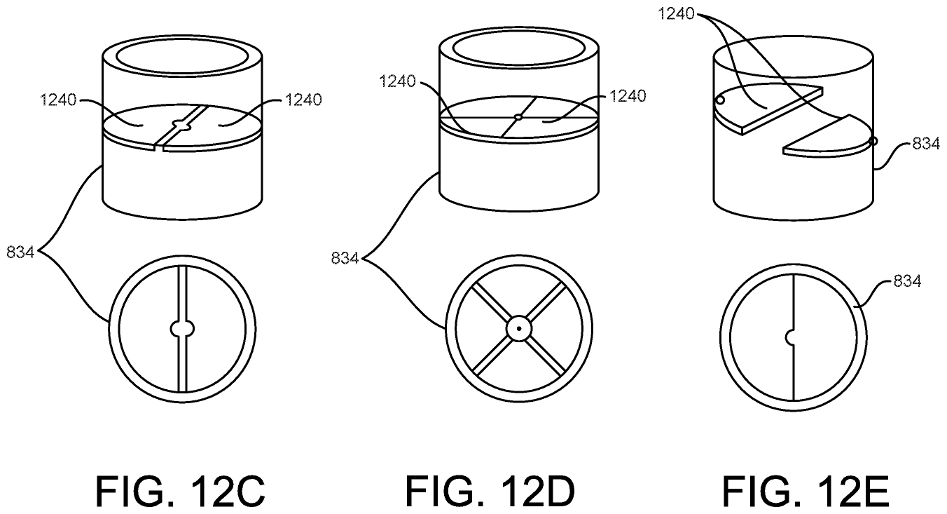

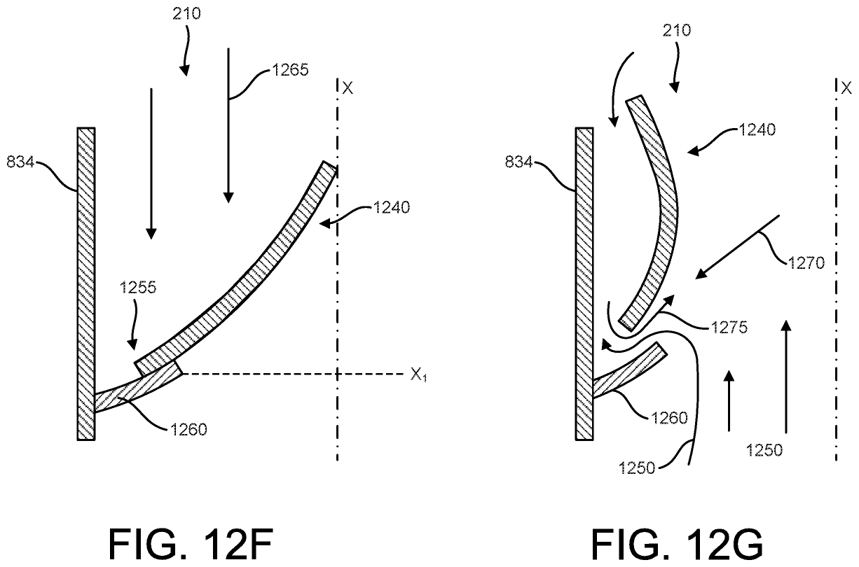

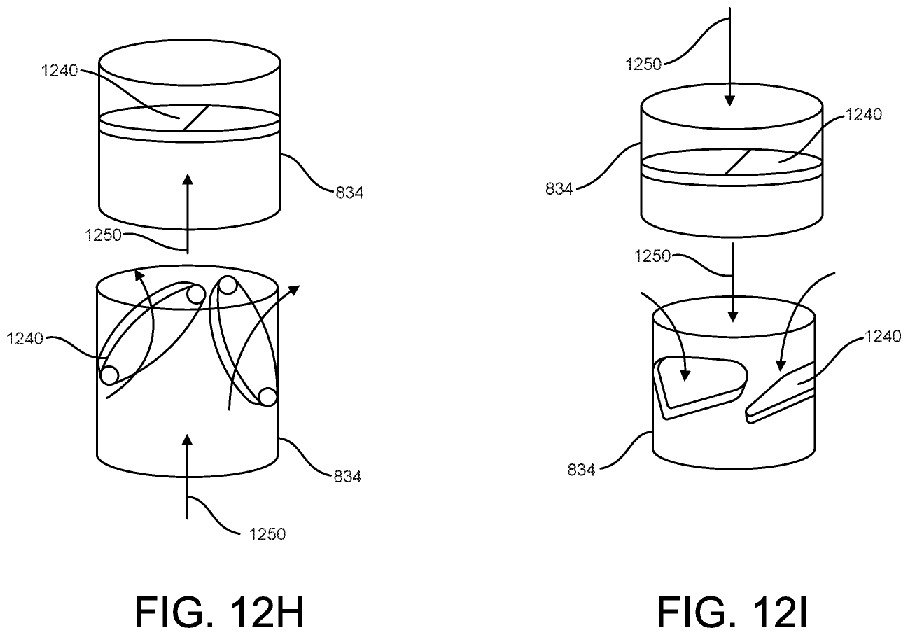

FIG. 12A is a side view and top view of another example indwelling flow restriction device 100 having a leaflet construct 1240, in accordance with various aspects of the present disclosure. The indwelling medical devices 100, as shown in FIGS. 12A-G, may be used for acute alteration of blood flow in an aorta or vena cava of a patient. The indwelling medical device 100 may also include a catheter 204, and a conduit 834 arranged at or extending from a distal end of the catheter 204. The conduit 834 includes a lumen 210 for the blood flow through the aorta or vena cava. In certain instances, the conduit 834 includes diametrically adjustable stent and/or graft components. In addition, the conduit 834 is configured to oppose against a vessel wall of the aorta or vena cava. The conduit 834 may include a self-expanding stent and/or graft and may expand against the vessel wall and secure to or oppose against the vessel wall. In certain instances, the conduit 834 may be a balloon structure.

The conduit 834 is arranged within the lumen 210 of the conduit 834. The leaflet construct 1240 is configured to alter the blood flow through the lumen 210 to restrict blood flow in the aorta or vena cava and alter blood flow into one or both kidneys of the patient. In certain instances, the leaflet construct 1240 may open and close in response to back flow pressure. The leaflet construct 1240 may be configured to restrict the blood flow at a desired percentage (e.g., to create a narrowed flow lumen in the conduit of the aorta distal of one or both renal arteries of between about 40% and about 80% to alter blood flow into the at least one branch vessel of the aorta or to create a narrowed flow lumen in the conduit located in the vena cava distal of one or both renal veins of between about 40% and about 90% to and alter blood flow through one or both of the renal veins).

In certain instances, the leaflet construct 1240 is coupled to one or more actuation members 1244 that are configured to open and close the leaflet construct 1240. In certain instances, the actuation members 1244 may be equal to a number of leaflets in the leaflet construct 1240, or a single actuation member 1244 may open and close each of the leaflets in the leaflet construct 1240. The actuation member 1244 may be configured to alter the blood flow through the lumen 210 to restrict blood flow in the aorta or vena cava and alter blood flow into one or both kidneys of the patient. In certain instances, the actuation member 1244 is configured to a level of restriction of blood flow through the lumen 210 based on the tension applied. In certain instances, the leaflet construct 1240 may be coupled to hinge members 1242 arranged within the lumen 210 to facilitate open and closed of the leaflet construct 1240. The actuation members 1244 members may be fibers, cords, metallic struts, semi-metallic struts, a super-elastic alloy material, or other similar structural elements. The actuation members 1244 may have a column strength that facilitates open and closing of the leaflet construct 1240 in response to a force or tension applied at ends of the actuation members 1244 not coupled to the leaflet construct 1240.

In various examples, the leaflet construct 1240 described herein may be formed of a biocompatible, synthetic material (e.g., including ePTFE and ePTFE composites, or other materials as desired). Other biocompatible polymers which can be suitable for use in synthetic leaflets include but are not limited to the groups of urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing. The leaflet construct 1240 may include an expandable fluoropolymer, used to form the expanded fluoropolymer material described, can comprise PTFE homopolymer. In other instances, blends of PTFE, expandable modified PTFE and/or expanded copolymers of PTFE can be used. Non-limiting examples of suitable fluoropolymer materials are described in, for example, U.S. Pat. No. 5,708,044, to Branca, U.S. Pat. No. 6,541,589, to Baillie, U.S. Pat. No. 7,531,611, to Sabol et al., U.S. patent application Ser. No. 11/906,877, to Bruchman, and U.S. patent application Ser. No. 12/410,050, to Xu et al. In addition, leaflet constructs 1240 are also described, for example, in U.S. Pat. No. 9,855,141 to Dienno et al.

In other examples, such leaflet construct is formed of a natural material, such as repurposed tissue, including bovine tissue, porcine tissue, or the like. The leaflet construct 1240 being of a synthetic material may facilitate the acute use of the device 100 as a non-synthetic leaflet construct 1240 may require a washing process. A synthetic leaflet construct 1240 does not require this additional step prior to use of the device 100.

The leaflet construct 1240 may be coupled or attached to the conduit 834. The term “coupled”, as used herein, means joined, connected, attached, adhered, affixed, or bonded. The leaflet(s) of the leaflet construct 1240 may be adhered (e.g., non-mechanically) to an exterior surface of the conduit 834 by an adhesive, thermal bonding, or chemical bonding. In this manner, the leaflet(s) of the leaflet construct 1240 are attached or coupled to the conduit 834 without puncturing or otherwise mechanically altering a surface of the leaflet(s) or the conduit 834 for attachment. In certain instances, the leaflets are attached, adhered, affixed, or bonded to the conduit 834 by an adhesive film. In certain instances, the leaflet construct 1240 may be incorporated within slits in the conduit 834. Reference may be made to U.S. application Ser. No. 16/129,673 to Colavito et al for discussion of leaflet attachment to a conduit 834.

In addition, an optional orifice 1246 may be arranged centrally between the leaflets of the leaflet construct 1240. The orifice 1246 may be sized to allow blood flow through the lumen 210 without totally occluding the aorta or vena cava. FIG. 12B is top down view of the indwelling flow restriction device 100 and the leaflet construct 1240, shown in FIG. 12A, in accordance with various aspects of the present disclosure.

In certain instances, the conduit 834 is arranged in a vessel other than the aorta or venal cava. In these instances, the leaflet construct 1240 may be configured to alter the blood flow through the lumen 210 to restrict blood flow in the vessel and induce a physiologically mediated therapeutic response in the patient. In certain instances, the leaflet construct 1240 is configured to induce the physiologically mediated therapeutic response to include an increase in peripheral resistance within the vessel. The leaflet construct 1240 may be configured to treat a fistula within the vessel and increase in peripheral resistance within the vessel as described in further detail below.

FIGS. 12C-E are side and top views of the indwelling flow restriction device 100, shown in FIG. 12A-B, with different leaflet construct 1240 arrangements, in accordance with various aspects of the present disclosure. As shown in FIG. 12C, the leaflets of the leaflet construct 1240 are arranged within the same plane. As shown in FIG. 12D, the leaflets of the leaflet construct 1240 include four leaflets. The leaflet construct 1240 may include two, three, four, five, six and additional numbers of leaflets. As shown in FIG. 12E, the leaflets of the leaflet construct 1240 are staggered longitudinally within the lumen 210.

FIG. 12F is a partial side view of the indwelling flow restriction device 100 and the leaflet construct 1240, shown in FIGS. 12A-E, when the leaflet construct 1240 is closed and FIG. 12G is a partial side view of the indwelling flow restriction device 100 and the leaflet construct 1240, shown in FIGS. 12A-F, when the leaflet construct 1240 is opened, in accordance with various aspects of the present disclosure.

In the closed position shown in FIG. 12F, the leaflet construct 1240 (a single leaflet is shown for ease of illustration) has a leaflet overlap region 1255 with a shelf 1260 preventing fluid flow through the lumen 210 in the retrograde direction 1265. The shelf 1260 may be arranged distal (relative to flow) behind the leaflet construct 1240 of any one of the arrangements shown in FIGS. 12A-E. During forward flow 1250 in the forward direction when the leaflet construct 1240 is not in the closed position (when the inflow pressure is greater than the outflow pressure), the leaflet construct 1240 moves away from the shelf 1260 to define a gap 1270 therebetween. Reference may be made to U.S. Patent Publication No. 2018/0353293 to Colavito et al. for further discussion of the gap 1270 and leaflet construct 1240 concept.

The gap 1270 formed between the leaflet construct 1240 and the shelf 1260 when the leaflet construct 1240 is not in the closed position allows fluid 1275 adjacent the leaflet construct 1240 to pass through the gap 1270 during forward flow 1250 in the forward direction through the lumen 210. That is, the recirculating flow behind the leaflet construct 1240 may pass through the gap 1270 preventing the recirculating flow from slowing down or stagnating behind the leaflet construct 1240. Further, the gap 1270 also allows forward flow 1250 to pass through the gap 1270 further disrupting and displacing the recirculating flow behind the leaflet construct 1240 to downstream of the leaflet construct 1240. Thus, blood behind the leaflet construct 1240 is less likely to clot or form thrombus.

FIG. 12H is side view of the indwelling flow restriction device 100 and the leaflet construct 1240, shown in FIGS. 12A-G, in response to venous flow and in accordance with various aspects of the present disclosure. For the venous implantation shown in FIG. 12H, the catheter (not shown) may be inserted in the femoral vein and tracked over a guidewire to a location distal to the renal vein (the other vena cava implantations discussed herein may also be deployed in this manner). In other instances, the device does not use a guidewire for delivery. In addition, the venous implantation may occur using an access path through the jugular vein. The leaflet construct 1240 may be opened and closed to allow blood flow 1250. The leaflet construct 1240 may be actuated to open the leaflet construct 1240 upwardly as shown.

FIG. 12I is side view of the indwelling flow restriction device 100 and the leaflet construct 1240, shown in FIGS. 12A-G, in response to arterial flow and in accordance with various aspects of the present disclosure. For the arterial implantation shown in FIG. 12I, the catheter (not shown) may be inserted in the iliac and tracked over a guidewire to a location distal to the renal arteries (the other aorta implantations discussed herein may also be deployed in this manner). The leaflet construct 1240 may be opened and closed to allow blood flow 1250. The leaflet construct 1240 may be actuated to open the leaflet construct 1240 downwardly as shown.

In each of these instances, the flow varies between systole and diastole. The leaflet construct 1240 may be configured for a design duty cycle via the actuation member 1244.

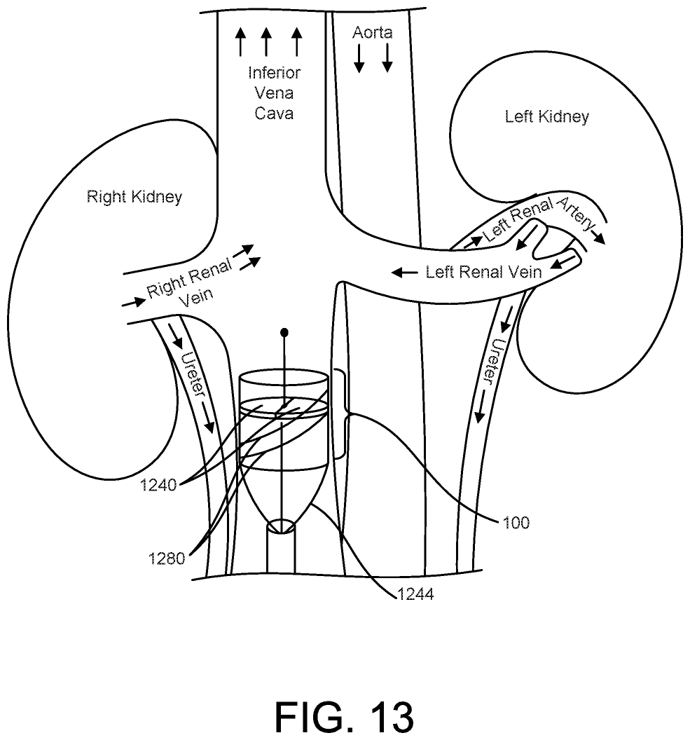

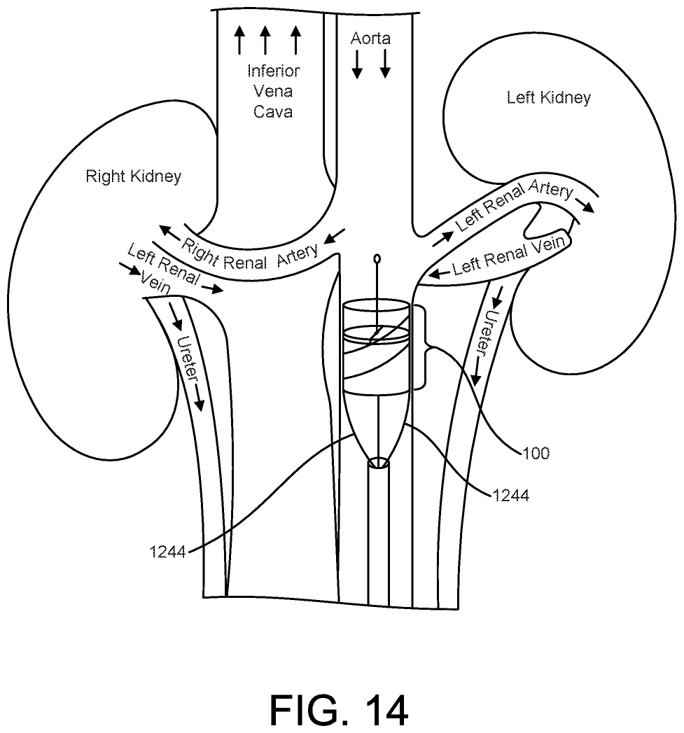

FIG. 13 is an illustration of the indwelling flow restriction device shown in FIGS. 12A-I arranged within a vena cava of a patient, in accordance with various aspects of the present disclosure. FIG. 14 is an illustration of the indwelling flow restriction device shown in FIGS. 12A-I arranged within an aorta of a patient, in accordance with various aspects of the present disclosure. As shown, the device may include constraining loops 1280 to facilitate delivery. The constraining loops 1280, similar to the constraining fibers 832a-c shown in FIG. 9A-B, may be used for delivery and/or for altering flow through the conduit 834.

When implanted in the aorta, the indwelling flow restriction device 100 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the indwelling flow restriction devices are configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the induced restriction is between about 50% and about 70% of a nominal flow.

When implanted in the vena cava, the indwelling flow restriction device 100 may augment perfusion from a tributary vessel (e.g., renal veins) terminating in the vena cava by altering pressure within the vena cava to alter blood flow from the tributary vessel of the vena cava. In certain instances, the indwelling flow restriction device 100 may be configured to create a narrowed flow lumen in the conduit located in the vena cava distal of the at least one tributary vessel of between about 40% and about 90%. Use of the indwelling flow restriction devices, discussed in further detail below, by dropping pressure in the renal veins may increase kidney perfusion hemodynamically rather than pharmaceutically.

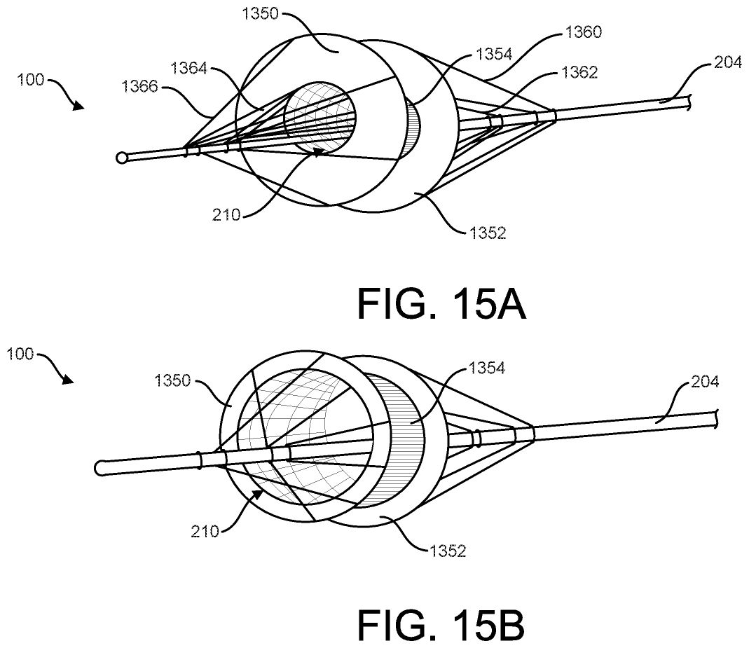

FIG. 15A is a side view of another example indwelling flow restriction device 100 in a first configuration, in accordance with various aspects of the present disclosure. FIG. 15B is a side view of the indwelling flow restriction device, shown in FIG. 15A, in a second configuration, in accordance with various aspects of the present disclosure. As noted above, the conduit 1354 is arranged between the upper portion 1350 and the lower portion 1352. The upper and lower portions 1350, 1352 may be configured to oppose against a vessel wall of the aorta or vena cava. In certain instances, the upper and lower portions 1350, 1352 are independently rotatable. Rotation of at least one of the upper and lower portions 1350, 1352 alters a diameter of the conduit 1354 and the lumen 210, which may restrict blood flow through the lumen 210. The upper and lower portions 1350, 1352 that are coupled to the catheter 204 by a plurality of struts 1360, 1362, 1364, 1366. As shown in FIGS. 14A-B, the upper and lower portions 1350, 1352 have a greater diameter than a central portion. In addition, the upper and lower portions 1350, 1352 are configured to contact and engage a vessel wall as is described in further detail below with reference to FIGS. 14A-B.

The struts 1360, 1362, 1364, 1366 may include separate sets of struts that are coupled to different portions along the catheter 204. For example, a first set of struts 1360 may be coupled at point “D” along the catheter 204, a second set of struts 1362 may be coupled at point “C” along the catheter 204, a third set of struts 1364 may be coupled at point “B” along the catheter 204, and a fourth set of struts 1366 may be coupled at point “A” along the catheter 204,

In certain instances, the sets of struts 1360, 1362, 1364, 1366 may be coupled to inner or outer sections of the upper and lower portions 1350, 1352. The first set of struts 1360 may be coupled to an outer circumference of the lower portion 1352 and the second set of struts 1362 may be coupled to an inner circumference of the lower portion 1352. In addition, the third set of struts 1364 may be coupled to an inner circumference of the upper portion 1350 and the fourth set of struts 1366 may be coupled to an outer circumference of the upper portion 1350.

Different portions of the catheter 204 may be independently rotatable to rotate one or both of the upper and lower portions 1350, 1352 by way of the sets of struts 1360, 1362, 1364, 1366. In certain instances, points A, B, C, and/or D of the catheter 204 may be configured to rotate. Rotation of these points on the catheter 204 pass the forces to sets of struts 1360, 1362, 1364, 1366 to rotate the desired upper and lower portions 1350, 1352. Rotation in one direction may restrict the conduit 1354 and rotation in the opposite direction may open the conduit 1354. Thus, the catheter 204 may be configured to alter the blood flow through the lumen 210 to restrict blood flow in the aorta or vena cava and alter blood flow into one or both kidneys of the patient.

In certain instances, the device 100 is arranged in a vessel other than the aorta or venal cava. In these instances, the upper and lower portions 1350, 1352 may be configured to alter the blood flow through the lumen 210 to restrict blood flow in the vessel and induce a physiologically mediated therapeutic response in the patient. In certain instances, the upper and lower portions 1350, 1352 is configured to induce the physiologically mediated therapeutic response to include an increase in peripheral resistance within the vessel. The upper and lower portions 1350, 1352 may be configured to treat a fistula within the vessel and increase in peripheral resistance within the vessel as described in further detail below.

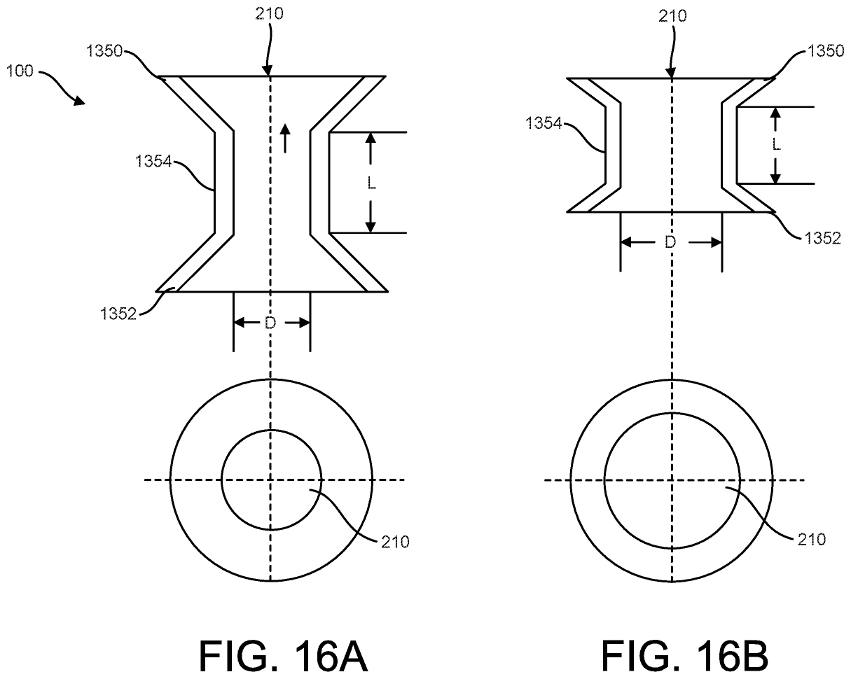

FIG. 16A is top and cross-sectional views of the indwelling flow restriction device 100, shown in FIGS. 15A-B, in the first configuration and FIG. 16B is a top and side view of the indwelling flow restriction device and upper and lower portions 1350, 1352, shown in FIGS. 15A-B, in the second configuration, in accordance with various aspects of the present disclosure. The indwelling medical devices 100, as shown in FIGS. 15A-B, may be used for acute alteration of blood flow in an aorta or vena cava of a patient. The indwelling medical device 100 may also include a catheter (not shown), and a conduit 1354 and upper and lower portions 1350, 1352 arranged at or extending from a distal end of the catheter. As discussed in detail above, the conduit 1354 is the portion of the device 100 that alters the restriction and changes diameter. The conduit 1354 and upper and lower portions 1350, 1352 include a lumen 210 for the blood flow through the aorta or vena cava. In certain instances, the conduit 1354 includes diametrically adjustable stent and/or graft components. In addition, the upper and lower portions 1350, 1352 are configured to contact the vessel wall of the aorta or vena cava and stabilize the conduit 1354 within the vessel. The conduit 1354 (and/or the upper and lower portions 1350, 1352) may include a self-expanding stent and/or graft and may expand against the vessel wall and secure to or oppose against the vessel wall. The stent portions within the conduit 1354 (and/or the upper and lower portions 1350, 1352) may be braided or formed from a cut-tube.

The conduit 1354 diameter and length are shown after rotation of at least one of the upper and lower portions 1350, 1352 for restriction in FIG. 15A in the first configuration and prior to rotation of at least one of the upper and lower portions 1350, 1352 in FIG. 15B. As shown comparing the conduit 1354 in these configurations, the diameter of the conduit 1354 decreases in response to rotation of at least one of the upper and lower portions 1350, 1352. In certain instances, the length of the conduit 1354 increases in response to rotation of at least one of the upper and lower portions 1350, 1352.

The upper and lower portions 1350, 1352 are configured to restrict the blood flow at a desired percentage (e.g., to create a narrowed flow lumen in the conduit of the aorta distal of one or both renal arteries of between about 40% and about 80% to alter blood flow into the at least one branch vessel of the aorta or to create a narrowed flow lumen in the conduit located in the vena cava distal of one or both renal veins of between about 40% and about 90% to and alter blood flow through one or both of the renal veins).

In certain instances and as described in further detail above with reference to FIGS. 15A-B, the catheter may be configured to rotate at least one of the upper portion 1350 and the lower portion 1352 to alter the blood flow through the lumen 210 to restrict blood flow in the aorta or vena cava and alter blood flow into one or both kidneys.

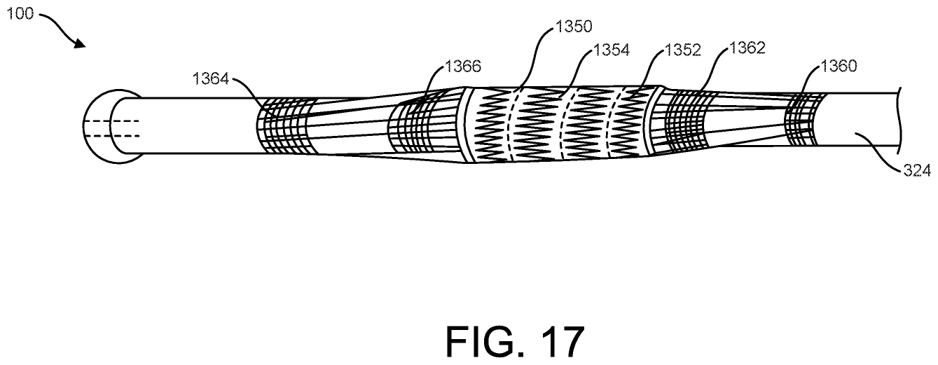

FIG. 17 is a side view of the indwelling flow restriction device 100, shown in FIGS. 15A-B and FIGS. 16A-B, in accordance with various aspects of the present disclosure. As shown in FIG. 17, the indwelling flow restriction device 100, such as the device 100 shown in FIGS. 15-16, is collapsed within a delivery sheath 324. The upper and lower portions 1350, 1352 and the conduit 1354 are arranged at delivery diameter.

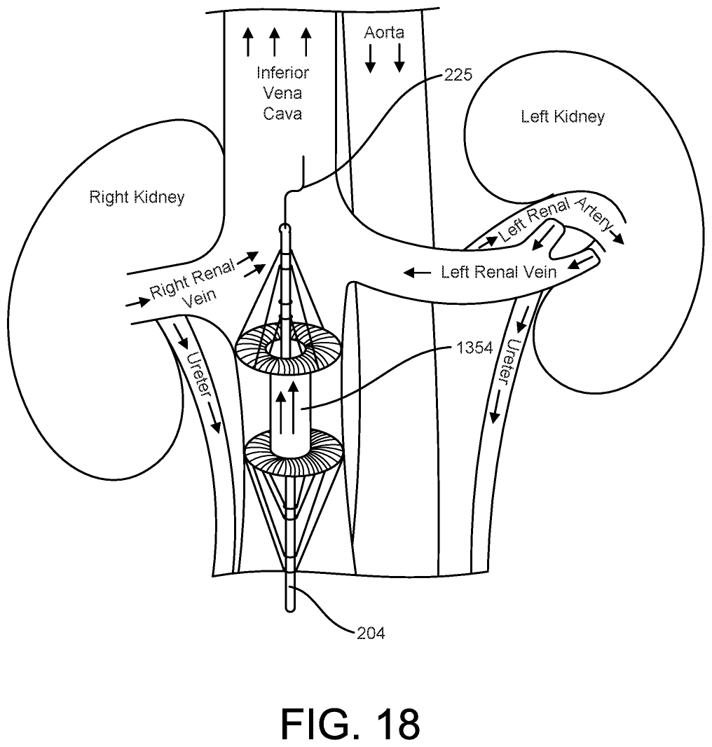

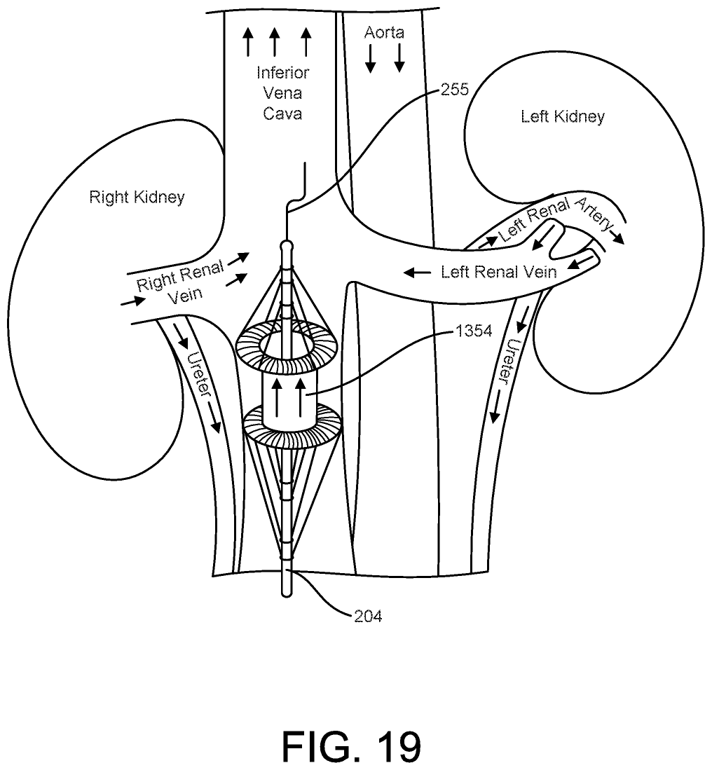

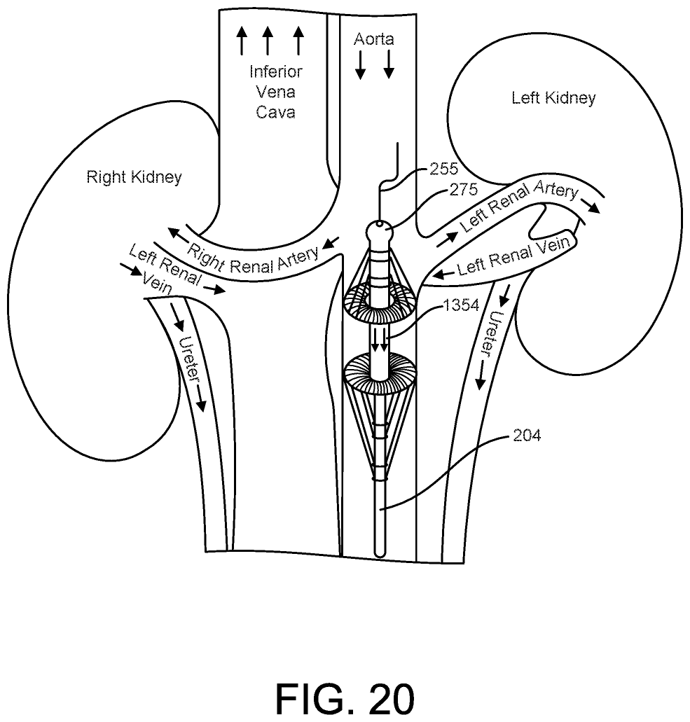

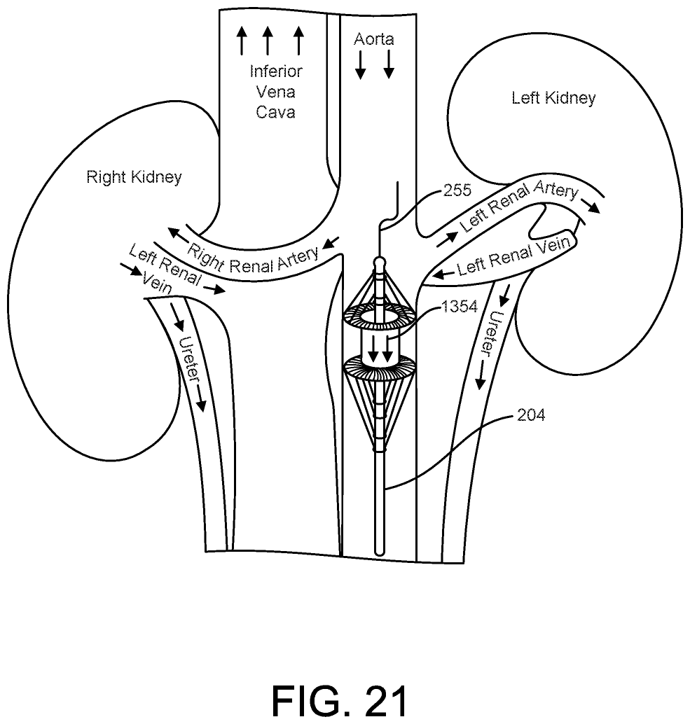

FIG. 18 is an illustration of the indwelling flow restriction device shown in FIGS. 15-17 in a first configuration arranged within a vena cava of a patient, in accordance with various aspects of the present disclosure FIG. 19 is an illustration of the indwelling flow restriction device shown in FIGS. 15-17 in a second configuration arranged within a vena cava of a patient, in accordance with various aspects of the present disclosure. FIG. 20 is an illustration of the indwelling flow restriction device shown in FIGS. 15-17 in a first configuration arranged within an aorta of a patient, in accordance with various aspects of the present disclosure. FIG. 21 is an illustration of the indwelling flow restriction device shown in FIGS. 15-17 in a second configuration arranged within an aorta of a patient, in accordance with various aspects of the present disclosure. In certain instances, the catheter 204 may include an atraumatic distal tip 275 as shown in FIG. 20.

When implanted in the aorta, the indwelling flow restriction device 100 is configured to redirect blood flow into at least one of the renal arteries by diverting fluid within the aorta. To achieve increased kidney perfusion, resistance to blood flow distal to the renal arteries may be increased, which decreases distal perfusion. The increased kidney perfusion enhances renal production and therefore removes fluid volume. In certain instances, the indwelling flow restriction devices are configured to create a narrowed flow lumen in the conduit of the aorta of the patient at least partially distal of the renal arteries between about 40% and about 80% and alter blood flow into at least one branch vessel of the aorta (e.g., one or both of the renal arteries). In certain instances, the narrowed flow lumen is between about 50% and about 70%.

When implanted in the vena cava, the indwelling flow restriction device 100 may augment perfusion from a tributary vessel (e.g., renal veins) terminating in the vena cava by altering pressure within the vena cava to alter blood flow from the tributary vessel of the vena cava. In certain instances, the indwelling flow restriction device 100 may be configured to create a narrowed flow lumen in the conduit located in the vena cava distal of the at least one tributary vessel of between about 40% and about 90%. Use of the indwelling flow restriction devices, discussed in further detail below, by dropping pressure in the renal veins may increase kidney perfusion hemodynamically rather than pharmaceutically.

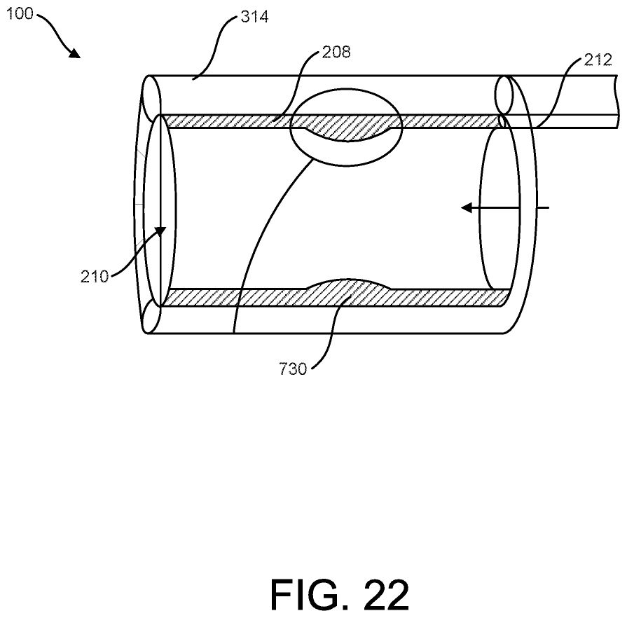

FIG. 22 is a side view of another example indwelling flow restriction device having a flow altering element, in accordance with various aspects of the present disclosure. The indwelling medical device 100, as shown in FIG. 22 may be used for acute alteration of blood flow in an aorta or vena cava of a patient. The indwelling medical device 100 may also include a catheter (not shown), and a vessel opposing member portion 314 arranged at or extending from a distal end of the catheter. The vessel opposing member portion 314 is configured to oppose against a vessel wall of the aorta or vena cava. As shown, the vessel opposing member portion 314 is an outer jacket (which may be formed from a graft material, a balloon, a polymer material, or another similar material, in part or whole). The vessel opposing member portion 314 is configured to oppose against a vessel wall of the aorta or vena cava. In certain instances, the outer jacket 314 is configured to expand and hold the device 100 against the vessel wall of the aorta or vena cava.

In certain instances, the flow altering element 208 is arranged within the vessel opposing member portion 314. As shown in FIG. 22, the flow altering element 208 is an inner jacket. The inner jacket 208 is includes a lumen 210 for directing the flow of blood that is pulsating through the aorta or vena cava. The inner jacket 208 may be configured to alter the blood flow through the lumen 210 to restrict blood flow in the aorta or vena cava and alter blood flow into one or both kidneys of the patient. In certain instances, the inner jacket 208, which may be a balloon or inflatable sheath, is configured to inflate and deflate to alter a level of restriction of blood flow through the lumen 210 by supplying and returning air, liquid, a super absorbent polymer (SAP), chemical reaction between polymers, non-Newtonian fluids, or another substance through an inflation port 212

After the indwelling flow restriction device 100 is secured or opposed, the flow altering element 208 may be selectively inflated and deflated (or restricted and unrestricted) to alter an amount of restriction of the lumen 210. A bladder 730 may be formed by the between the inner jacket 208 and the outer jacket 314. that is consistent over the entire circumference (360 degrees) of the internal surfaces of the flow altering element 208 or formed in portions or sections (e.g., pockets spaced 180 degrees) of the internal surfaces of the flow altering element 208.

In certain instances, the flow altering element 208 is arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

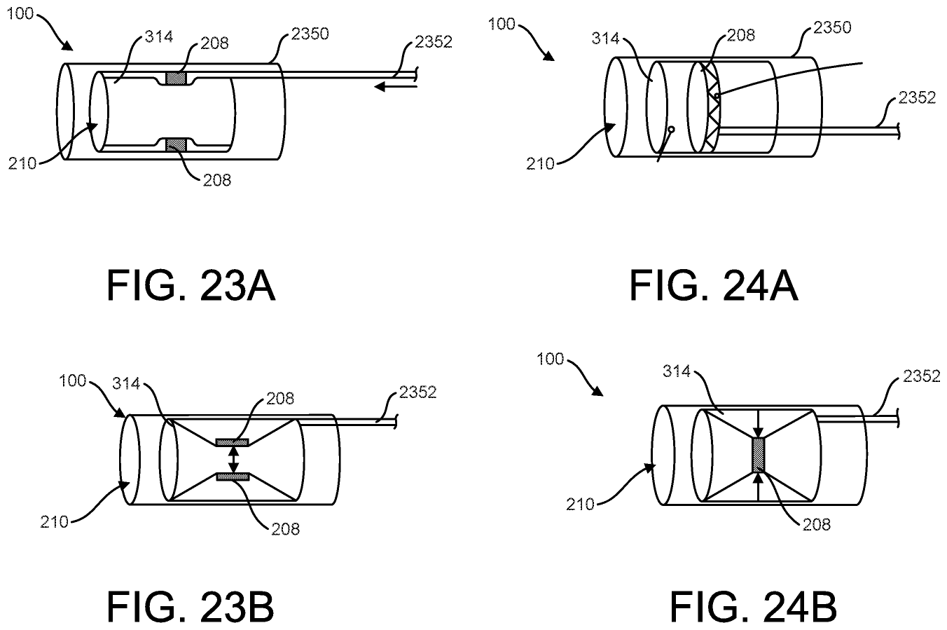

FIG. 23A is a side view of another example indwelling flow restriction device 100 in a first configuration, in accordance with various aspects of the present disclosure. The indwelling medical device 100, as shown in FIGS. 23A-B may be used for acute alteration of blood flow in an aorta or vena cava of a patient. The indwelling medical device 100 may also include a catheter (not shown), and a vessel opposing member portion 314 arranged at or extending from a distal end of the catheter. The vessel opposing member portion 314 is configured to oppose against a vessel wall 2350 of the aorta or vena cava.

In certain instances, a flow altering element 208 is arranged outside of the vessel opposing member portion 314 to constrict at least a portion of the vessel opposing member portion 314 as shown in comparing FIGS. 23A and 23B. The flow altering element 208 may be one or more magnets arranged about the circumference of the vessel opposing member portion 314. The magnets, as the flow altering element 208, may be actuated by way of an actuator 2352. The actuator 2352 may be configured to activate the magnets to attract together to constrict the vessel opposing member portion 314. In this manner, the magnets alter a lumen 210 of the vessel opposing member portion 314 to direct the flow of blood that is pulsating through the aorta or vena cava. The magnets may be configured to alter the blood flow through the lumen 210 to restrict blood flow in the aorta or vena cava and alter blood flow into one or both kidneys of the patient. After the indwelling flow restriction device 100 is secured or opposed, the magnets may be selectively activated to alter an amount of restriction of the lumen 210.

In certain instances, the flow altering element 208 is arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

FIG. 24A is a side view of another example indwelling flow restriction device in a first configuration, in accordance with various aspects of the present disclosure. The indwelling medical device 100, as shown in FIGS. 24A-B may be used for acute alteration of blood flow in an aorta or vena cava of a patient. The indwelling medical device 100 may also include a catheter (not shown), and a vessel opposing member portion 314 arranged at or extending from a distal end of the catheter. The vessel opposing member portion 314 is configured to oppose against a vessel wall 2350 of the aorta or vena cava.

In certain instances, a flow altering element 208 is arranged outside of the vessel opposing member portion 314 to constrict at least a portion of the vessel opposing member portion 314 as shown in comparing FIGS. 24A and 24B. The flow altering element 208 may be a cinching band arranged about the circumference of the vessel opposing member portion 314. The cinching band, as the flow altering element 208, may be actuated by way of an actuator 2352. In certain instances, the cinching band may include a pathway for a polymer or activatable substance (e.g., an electro-active polymer) that expands in diameter within the cinching band, which thereby restricts or constricts a diameter of at least a portion of the vessel opposing member portion 314. Thus, the cinching band is configured to restrict or constrict a diameter of at least a portion of the vessel opposing member portion 314 in response to activation of the polymer or activatable substance (e.g., an electro-active polymer).

The actuator 2352 may be configured to activate the polymer or activatable substance (e.g., an electro-active polymer) to constrict the vessel opposing member portion 314. In this manner, the cinching band alters a lumen 210 of the vessel opposing member portion 314 to direct the flow of blood that is pulsating through the aorta or vena cava. The cinching band may be configured to alter the blood flow through the lumen 210 to restrict blood flow in the aorta or vena cava and alter blood flow into one or both kidneys of the patient. After the indwelling flow restriction device 100 is secured or opposed, the cinching band may be selectively activated to alter an amount of restriction of the lumen 210.

In certain instances, the flow altering element 208 is arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

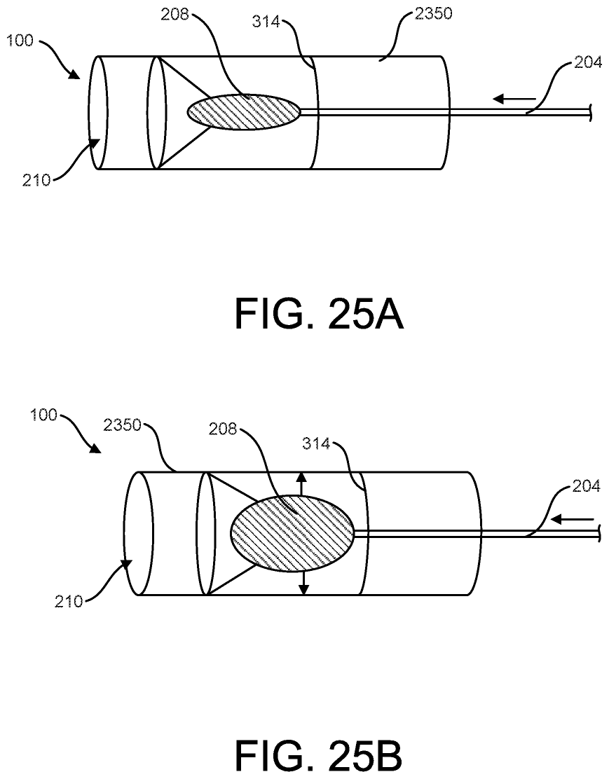

FIG. 25A is a side view of another example indwelling flow restriction device in a first configuration, in accordance with various aspects of the present disclosure. The indwelling medical device 100, as shown in FIGS. 25A-B may be used for acute alteration of blood flow in an aorta or vena cava of a patient. The indwelling medical device 100 may also include a catheter 204, and a vessel opposing member portion 314 (e.g., a graft or stent-graft) arranged at or extending from a distal end of the catheter. The vessel opposing member portion 314 is configured to oppose against a vessel wall 2350 of the aorta or vena cava.

In certain instances, a flow altering element 208 is arranged within the vessel opposing member portion 314 to expand to restrict a lumen 210 of the vessel opposing member portion 314 as shown in comparing FIGS. 24A and 24B. The flow altering element 208 may be a balloon arranged centrally within the vessel opposing member portion 314. Inflation of the balloon creates a funnel shape of at a leading end of the vessel opposing member portion 314. In certain instances, the vessel opposing member portion 314 is coupled to at least a portion of the balloon as is shown. In this manner, inflation of the balloon draws portions of the vessel opposing member portion 314 inwardly to restrict the lumen 210.

In certain instances, the flow altering element 208 is arranged in a vessel other than the aorta or venal cava. In these instances, the flow altering element 208 may be configured to alter the blood flow through the lumen 210 to restrict blood flow in the vessel and induce a physiologically mediated therapeutic response in the patient. In certain instances, the flow altering element 208 is configured to induce the physiologically mediated therapeutic response to include an increase in peripheral resistance within the vessel. The flow altering element 208 may be configured to treat a fistula within the vessel and increase in peripheral resistance within the vessel as described in further detail below.

The above figures illustrate devices that may be used for acute treatment through implantation in a vessel to increased blood flow into branch vessels of the vessel into which the devices are arranged (e.g., the aorta and/or vena cava and branch vessels extending from or leading to the aorta and/or vena cava). In certain instances, the devices may be actuated to alter blood flow using a duty cycle. The duty cycle may sync with systolic and diastolic blood flow pressure. In other instances, the duty cycle may be based off of other physiological signals or randomly selected. In addition, the amount of restriction may be altered based on a patient's activity level (e.g., standing up, sitting down, sleeping, exercising, at-rest) to tailor blood flow without cutting off circulation below the restriction location. A sensor, as described above, may sense the activity level and provide feedback to alter the restriction percentage.

In addition and in certain instances, the devices discussed above describe diametric restriction of the aorta or vena cava, however, a length of the restriction portion also may affect the amount of restriction in the aorta or vena cava. Thus, the length of the restriction portion and the diameter or circumference of the restriction portion may be varied to achieve a desired stenosis or restriction percentage.

Further, the devices discussed herein may implanted within vessels for treatment of an arteriovenous (AV) fistula. AV fistula formation may lead to a decrease in peripheral resistance within the vasculature. Implantation of the devices discussed herein at or adjacent to the AV fistula may increase flow resistance to a nominal level and counteract the decrease in peripheral resistance resulting from the AV fistula. In certain instances, the devices are implanted distal to the AV fistula, proximal to the AV fistula, or across the AV fistula.

### Test Methods

A non-limiting example of a suitable test method for the flow restriction devices and methods involved evaluating response of canines with induced heart failure (coronary microembolization resulting in ejection fraction of about 30%). Four canines received flow restricting devices distal to the renal arteries and three were kept for controls. Average diameter stenosis induced by the devices was about 60% (range of about 55-about 64%). Animals were kept in life for 35 days; blood chemistry, biomarkers, and hemodynamic status were monitored throughout the study.

Shifts in hemodynamic status were observed in the test group relative to the control group indicating improved cardiac function and decreased sympathetic nervous system tone. For example, heart rate and mean arterial pressure decreased, while contractility increased relative to controls. These comparative outcomes were supported by positive shifts in biomarkers such as pro-BNP and NGAL, relative to controls. As an example, animals with implant produced about 35% more urine with about 21% higher creatinine content, resulting in about 52% less increase in serum creatinine as a result of the diuretic challenge. Table 1 shows the results for the study.

TABLE 1
Study Results

| | | | 35 days post | | Change during | |
|---|---|---|---|---|---|---|
| | pre-implant | | implant | | study | |
| | test | control | test | control | test | control |
| Mean Rate | 86 | 86 | 80 | 90 | −6 | 4 |
| (bpm) | | | | | | |
| Mean Arterial | 74 | 71 | 65 | 69 | −9 | −2 |
| Pressure | | | | | | |
| (mmHg) | | | | | | |
| Mean Heart | 1384 | 1367 | 1581 | 1299 | 197 | −68 |
| Contractility | | | | | | |
| (mmHg/s) | | | | | | |
| Mean | 1442 | 1501 | 1233 | 1699 | −209 | 198 |
| nt-proBNP | | | | | | |
| Biomarker | | | | | | |
| (pg/ml)Mean | | | | | | |
| Plasma NGAL | 5.5 | 6.22 | 6.14 | 8.62 | 0.64 | 2.4 |
| Biomarker | | | | | | |
| (ng/ml) | | | | | | |

At the end of the study, all animals (test and control) received an 80 mg bolus of Lasix (a diuretic). In this model healthy animals experience minimal change in serum creatinine levels while animals in heart failure experience a significant increase. This test was conducted to illustrate the impact of the device when the kidney is stressed. Table 2 shows the post-bolus results.

TABLE 2
Post-Bolus Results

| | | | 4 hours post- | | Change during | |
|---|---|---|---|---|---|---|
| | pre-diuretic | | diuretic | | study | |
| | test | control | test | control | test | control |
| Mean Serum | 0.9 | 0.77 | 1.05 | 1.08 | 0.15 | 0.31 |
| Creatinine | | | | | | |
| Mean Urine | | | 46 | 38 | | |
| Creatinine | | | | | | |
| (mg/dL) | | | | | | |
| Mean Urine | | | 348 | 258 | | |
| Output (ml) | | | | | | |

The flow restricting devices, as discussed in detail above, may achieve similar results when placed in the aorta distal to the renal arteries to help decrease the symptoms of fluid overload and cardiac stress associated with heart failure. The devices may increase blood pressure proximal to the stenosis and, in doing so, increase kidney perfusion pressure, thus increasing kidney perfusion. A secondary effect of this device may be a reduction in the activation of the RAAS system. Effectiveness of the device was based on assessment of central hemodynamics, left ventricular (LV) function, renal function and biomarkers.

In certain instances, induced stenosis may have little effect on flow or pressure until it reaches about 40%, after which the impact is dependent on artery diameter and blood flow rate. The regime for stenosis may be between about 40% and about 80% or between about 50% and about 70%. Clinically, measurement of ankle pressure, Doppler ultrasound velocity, or other hemodynamic parameters in the lower limbs can be employed to optimize the magnitude of the induced stenosis while ensuring adequate limb perfusion.

The illustrative indwelling medical devices may include diametrically adjustable stent and/or graft components. For example, the stent components, discussed herein, may be diametrically adjustable such that elongation of the device may increase a diameter of the stent component, and shortening of the device may decrease a diameter of the stent component. For further discussion regarding diametrically adjustable stent components, reference may be made to U.S. Pat. No. 8,936,634, for the teachings of diametrically adjustable stent components. In addition, the graft components discussed herein may also be diametrically adjustable. For further discussion regarding diametrically adjustable graft components, reference may be made to U.S. Pat. Nos. 6,336,937 and 9,522,072, for the teachings of diametrically adjustable graft components. In addition, the indwelling medical devices may alter flow into the kidneys and produce a neuro-hormonal response that effects a change in the patient as discussed in further detail with reference to FIG. 1.

Examples of synthetic polymers (which may be used as a graft component) include, but are not limited to, nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyamides, their mixtures, blends and copolymers are suitable as a graft material. In one embodiment, said graft is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON®. or GORE-TEX®), and porous or nonporous polyurethanes. In certain instances, the graft comprises expanded fluorocarbon polymers (especially PTFE) materials described in British. Pat. No. 1,355,373; 1,506,432; or 1,506,432 or in U.S. Pat. No. 3,953,566; or 4,187,390. Included in the class of preferred fluoropolymers are polytetrafluoroethylene (PTFE), fluorinated ethylene propylene (FEP), copolymers of tetrafluoroethylene (TFE) and perfluoro(propyl vinyl ether) (PFA), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinylfluoride (PVF). Especially preferred, because of its widespread use in vascular prostheses, is ePTFE. In certain instances, the graft comprises a combination of said materials listed above. In certain instances, the graft is substantially impermeable to bodily fluids. Said substantially impermeable graft can be made from materials that are substantially impermeable to bodily fluids or can be constructed from permeable materials treated or manufactured to be substantially impermeable to bodily fluids (e.g. by layering different types of materials described above or known in the art).

Additional examples of graft materials include, but are not limited to, vinylidinefluoride/hexafluoropropylene, hexafluoropropylene (HFP), tetrafluoroethylene (TFE), vinylidenefluoride, 1-hydropentafluoropropylene, perfluoro(methyl vinyl ether), chlorotrifluoroethylene (CTFE), pentafluoropropene, trifluoroethylene, hexafluoroacetone, hexafluoroisobutylene, fluorinated poly(ethylene-co-propyl ene (FPEP), poly(hexafluoropropene) (PHFP), poly(chlorotrifluoroethylene) (PCTFE), poly(vinylidene fluoride (PVDF), poly(vinylidene fluoride-co-tetrafluoroethylene) (PVDF-TFE), poly(vinylidene fluoride-co-hexafluoropropene) (PVDF-HFP), poly(tetrafluoroethylene-co-hexafluoropropene) (PTFE-HFP), poly(tetrafluoroethylene-co-vinyl alcohol) (PTFE-VAL), poly(tetrafluoroethylene-co-vinyl acetate) (PTFE-VAC), poly(tetrafluoroethylene-co-propene) (PTFEP) poly(hexafluoropropene-co-vinyl alcohol) (PHFP-VAL), poly(ethylene-co-tetrafluoroethylene) (P ETFE), poly(ethylene-co-hexafluoropropene) (PEHFP), poly(vinylidene fluoride-co-chlorotrifluoroe-ethylene) (PVDF-CTFE), and combinations thereof, and additional polymers and copolymers described in U.S. Publication 2004/0063805. Additional polyfluorocopolymers include tetrafluoroethylene (TFE)/perfluoroalkylvinylether (PAVE). PAVE can be perfluoromethylvinylether (PMVE), perfluoroethylvinylether (P EVE), or perfluoropropylvinylether (PPVE), as essentially described in U.S. Publication 2006/0198866 and U.S. Pat. No. 7,049,380. Other polymers and copolymers include, polylactide, polycaprolacton-glycolide, polyorthoesters, polyanhydrides; poly-am inoacids; polysaccharides; polyphosphazenes; poly(ether-ester) copolymers, e.g., PEO-PLLA, or blends thereof, polydim ethyl-siolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters and any polymer and copolymers described in U.S. Publication 2004/0063805.

The graft components, as discussed herein, may be attached to the self-expanding stent elements by using a coupling member that is generally a flat ribbon or tape having at least one generally flat surface. In certain instances, the tape member is made from expanded PTFE (ePTFE) coated, continuously or discontinuously, with an adhesive. The adhesive may be a thermoplastic adhesive. In certain instances, the thermoplastic adhesive may be fluorinated ethylene propylene (FEP). More specifically, an FEP-coated side of the ePTFE may face toward and contacts an exterior surface of the self-expanding stent and graft component, thus attaching the self-expanding stent to the graft component. Materials and method of attaching a stent to the graft is discussed in U.S. Pat. No. 6,042,605.

The stent elements discussed herein can be fabricated from a variety of biocompatible materials. These materials may include 316L stainless steel, cobalt-chromium-nickel-molybdenum-iron alloy (“cobalt-chromium”), other cobalt alloys such as L605, tantalum, nickel-titanium alloys (e.g., Nitinol), or other biocompatible metals. In certain instances, as discussed in detail above, the stent (and graft) may be self-expanding. The prosthesis may be balloon expandable or self-expanding, or non-metal (e.g., PEEK, 3-D printed materials, PLA).

A variety of materials variously metallic, super elastic alloys, such as Nitinol, are suitable for use in these stents. Primary requirements of the materials are that they be suitably springy even when fashioned into very thin sheets or small diameter wires. Various stainless steels which have been physically, chemically, and otherwise treated to produce high springiness are suitable as are other metal alloys such as cobalt chrome alloys (e.g., ELGILOY®), platinum/tungsten alloys, and especially the nickel-titanium alloys (e.g., Nitinol).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. An indwelling medical device for acute alteration of blood flow in a vessel of a patient, the device comprising:
a catheter;
a vessel opposing member portion arranged at or extending from a distal end of the catheter and configured to oppose against a vessel wall of the vessel; and
a flow altering element, arranged within the vessel opposing member portion and including a lumen for the blood flow through the vessel therethrough, configured to alter the blood flow through the lumen to restrict blood flow in the vessel and induce a physiologically mediated therapeutic response in the patient.

2. The device of claim 1, wherein the vessel is the aorta and the vessel opposing member portion is configured to oppose against the vessel wall in the aorta and create a narrowed flow lumen in a conduit located in the aorta distal of one or both renal arteries of between about 40% and about 80% to alter blood flow into the at least one branch vessel of the aorta to alter blood flow into one or both kidneys.

3. The device of claim 1, wherein the vessel is the vena cava and the vessel opposing member portion is configured to oppose against the vessel wall in the vena cava create a narrowed flow lumen in the conduit located in the vena cava distal of one or both renal veins of between about 40% and about 90% to and alter blood flow through one or both of the renal veins to alter blood flow into one or both kidneys.

4. The device of claim 3, wherein the vessel opposing member portion is arranged upstream from one or both of the renal veins and the flow altering element is configured to drop blood pressure out of one or both of the renal veins to promote blood flow through the kidneys.

5. The device of claim 1, wherein the flow altering element is at least one of a balloon, stent, stent-graft, sheath, and a restricting band, and vessel opposing member portion is comprised of at least one or more elements such as an outer balloon, a stent, a polymer material or other structures.

6. The device of claim 5, wherein the flow altering element is a balloon, and the vessel opposing member portion is at least one of an outer balloon and further including a plurality of inflation ports configured to inflate and deflate the outer balloon and the flow altering element.

7. The device of claim 6, wherein the outer balloon and the flow altering element are of substantially similar lengths.

8. The device of claim 5, wherein a length of the flow altering element is less than a length of the outer balloon.

9. The device of claim 6, wherein one of the plurality of ports is configured to provide flow to restrict an internal diameter of at least a portion of the outer balloon to alter the lumen.

10. The device of claim 6, further including a plurality of struts coupled to the catheter and arranged within the inner or outer balloon, the plurality of struts being configured to support the balloon.

11. The device of claim 1, wherein the lumen of the flow altering element includes at least one of a cylindrical shape, hourglass shape, a shape with circular restriction portions, a shape with stepped restriction portions, a ramped shape, and a peaked shape.

12. The device of claim 1, wherein the flow altering element is at least one of a balloon, stent, stent-graft, restricting band, and sheath.

13. A method of increasing blood flow into one or booth kidneys of the patient to increase urine production including the device of claim 1.

14. An indwelling medical device for acute alteration of blood flow in an aorta or vena cava of a patient, the device comprising:
a catheter;
a conduit, arranged at or extending from a distal end of the catheter, including a lumen for the blood flow through the aorta or vena cava; and
at least one constraining fiber, arranged about a circumference of the conduit, configured to alter the blood flow through the lumen to restrict blood flow in the aorta or vena cava and alter blood flow into one or both kidneys of the patient in response to tension.

15.-19. (canceled)

20. An indwelling medical device for acute alteration of blood flow in an aorta or vena cava of a patient, the device comprising:
a catheter;
a conduit, arranged at or extending from a distal end of the catheter, including a lumen for the blood flow through the aorta or vena cava; and
a leaflet construct, arranged within the lumen of the conduit, configured to alter the blood flow through the lumen to restrict blood flow in the aorta or vena cava and alter blood flow into or out of one or both kidneys of the patient.

21. The device of claim 20, wherein the conduit is configured to oppose against the vessel wall in the aorta and create a narrowed flow lumen of the aorta distal of one or both renal arteries of between about 40% and about 80% to promote blood flow to the kidneys.

22. The device of claim 20, wherein the conduit is configured to oppose against the vessel wall in the vena cava and create a narrowed flow lumen of the vena cava distal of one or both renal veins of between about 40% and about 90% to promote blood flow into the kidney.

23. The device of claim 22, wherein the conduit is arranged upstream from one or both of the renal veins and the leaflet construct is configured to drop blood pressure out of one or both of the renal veins to promote blood flow through the kidneys.

24. The device of claim 20, further including one or more actuation members that are configured to open and close the leaflet construct.

25. The device of claim 24, wherein the one or more actuation members includes a single actuation member configured to open and close each of the leaflets in the leaflet construct.

26. The device of claim 24, wherein the one or more actuation members includes a number of actuation members equal to a number of leaflets in the leaflet construct.

27. (canceled)

28. A method of increasing blood flow into or out of one or booth kidneys of the patient to increase urine production including the device of claim 20.

29. An indwelling medical device for acute alteration of blood flow in an aorta or vena cava of a patient, the device comprising:
an upper portion configured to oppose against a vessel wall of the aorta or vena cava;
a lower portion configured to oppose against a vessel wall of the aorta or vena cava;
a conduit, arranged between the upper portion and the lower portion, including a lumen for the blood flow through the aorta or vena cava; and
a catheter configured to rotate at least one of the upper portion and the lower portion to alter the blood flow through the lumen to restrict blood flow in the aorta or vena cava and alter blood flow into one or both kidneys.

30. The device of claim 29, wherein the upper portion and the lower portion are configured to oppose against the vessel wall in the aorta and create a narrowed flow lumen in the conduit located in the aorta, distal of one or both renal arteries of between about 40% and about 80% to increase blood flow to the kidneys.

31. The device of claim 29, wherein the upper portion and the lower portion are configured to oppose against the vessel wall in the vena cava to create a narrowed flow lumen in the conduit located in the vena cava, distal of one or both renal veins of between about 40% and about 90% to and alter blood flow through one or both of the renal veins.

32. The device of claim 31, wherein the upper portion and the lower portion are arranged upstream from one or both of the renal veins and configured to drop blood pressure out of one or both of the renal veins to promote blood flow through the kidneys.

33. The device of claim 29, further comprising a first plurality of struts coupled to the catheter and the upper portion configured to rotate the upper portion in response to rotation of the catheter, and a second plurality of struts coupled to the catheter and the lower portion configured to rotate the lower portion in response to rotation of the catheter.

34. The device of claim 29, wherein the catheter is configured to independently rotate the upper portion and the lower portion.

35. The device of claim 30, wherein the conduit is substantially cylindrical and includes a diameter less than a diameter of at least one of the upper portion and the lower portion.

36. A method of increasing blood flow into one or booth kidneys of the patient to increase urine production including the device of claim 29.

37. A method of acute treatment of heart failure of a patient, the method comprising:
arranging an indwelling medical device coupled to a catheter within an aorta or vena cava of the patient;
restricting blood flow through the indwelling medical device to induce a physiologically mediated therapeutic response by reducing buildup of excess fluid within the patient; and
removing the indwelling medical device from the aorta or vena cava of the patient.

38.-49. (canceled)

50. A device for restricting blood flow within a vessel, the device comprising:
an indwelling medical device coupled to a catheter configured to implant within the vessel configured to restrict blood flow through the indwelling medical device to induce a physiologically mediated therapeutic response.

51. The device of claim 50, wherein the vessel is the aorta or vena cava, and the indwelling medical device includes a vessel opposing member portion arranged at or extending from a distal end of the catheter and configured to oppose against a vessel wall of the aorta or vena cava and a flow altering element, arranged within the vessel opposing member portion and including a lumen for the blood flow through the aorta or vena cava therethrough, configured to alter the blood flow through the lumen to restrict blood flow in the aorta or vena cava and alter blood flow into one or both kidneys of the patient.

52. The device of claim 50, wherein the vessel is the aorta or vena cava, and the indwelling medical device includes a conduit, arranged at or extending from a distal end of the catheter, including a lumen for the blood flow through the aorta or vena cava; and at least one constraining fiber, arranged about a circumference of the conduit, configured to alter the blood flow through the lumen to restrict blood flow in the aorta or vena cava and alter blood flow into one or both kidneys of the patient in response to tension.

53. The method of claim 50, wherein the vessel is the aorta or vena cava, and the indwelling medical device includes a conduit, arranged at or extending from a distal end of the catheter, including a lumen for the blood flow through the aorta or vena cava; and a leaflet construct, arranged within the lumen of the conduit, configured to alter the blood flow through the lumen to restrict blood flow in the aorta or vena cava and alter blood flow into one or both kidneys of the patient.

54. The device of claim 50, wherein the vessel is the aorta or vena cava, and the indwelling medical device includes an upper portion configured to oppose against a vessel wall of the aorta or vena cava; a lower portion configured to oppose against a vessel wall of the aorta or vena cava; a conduit, arranged between the upper portion and the lower portion, including a lumen for the blood flow through the aorta or vena cava; and the catheter is configured to rotate at least one of the upper portion and the lower portion to alter the blood flow through the lumen to restrict blood flow in the aorta or vena cava and alter blood flow into one or

55. The device of claim 50, wherein the vessel is the aorta or vena cava, and the indwelling medical device includes a vessel opposing member portion, arranged at or extending from a distal end of the catheter, including a lumen for the blood flow through the aorta or vena cava, and at least one magnet configured to alter a diameter of the lumen to direct the flow of blood that is pulsating through the aorta or vena cava.

56. The device of claim 50, wherein the vessel is the aorta or vena cava, and the indwelling medical device includes a vessel opposing member, arranged at or extending from a distal end of the catheter, including a lumen for the blood flow through the aorta or vena cava, and a cinching band is configured to restrict or constrict a diameter of the vessel opposing member in response to activation of the cinching band.

57. The device of claim 50, wherein the vessel is the aorta or vena cava, and the indwelling medical device includes a vessel opposing member, arranged at or extending from a distal end of the catheter, including a lumen for the blood flow through the aorta or vena cava, and a cinching band including an activatable substance that is configured to restrict or constrict a diameter of the vessel opposing member in response to activation of the activatable substance.

58. The device of claim 50, wherein the vessel is the aorta or vena cava, and the indwelling medical device includes a vessel opposing member, arranged at or extending from a distal end of the catheter, including a lumen for the blood flow through the aorta or vena cava, and an actuator that is configured to restrict or constrict a diameter of the vessel opposing member in response to activation of the actuator.

59. The device of claim 50, wherein the vessel is the aorta or vena cava, and the indwelling medical device includes a vessel opposing member, arranged at or extending from a distal end of the catheter, including a lumen for the blood flow through the aorta or vena cava, and a balloon configured to create a funnel shape at a leading end of the vessel opposing member portion.
